(19) United States
(12) Patent Application Publication
(10) Pub. No.: US 2022/0378697 A1
(43) Pub. Date: Dec. 1, 2022

(54) SUPPOSITORY CAPSULE

(71) Applicant: R.P. Scherer Technologies, LLC, Carson City, NV (US)

(72) Inventors: José Rey, Indian Rocks Beach, FL (US); William M. Bell, Lutz, FL (US)

(21) Appl. No.: 17/772,695

(22) PCT Filed: Oct. 30, 2020
(86) PCT No.: PCT/US2020/058126

§ 371 Date: Apr. 28, 2022

Related U.S. Application Data
(60) Provisional application No. 62/928,679, filed on Oct. 31, 2019.

(51) Int. Cl. A61K 9/02; A61J 3/08

(52) U.S. Cl. CPC A61K 9/025; A61J 3/08

(57) ABSTRACT

Disclosed is a suppository comprising a suppository base. The suppository may have a length, relative to a horizontal axis, that is greater than its width, relative to a vertical axis. The suppository may have a proximal end and a distal end along the horizontal axis. The proximal end may be adapted for insertion into a body cavity. The distal end may have at least one indentation and adapted to be gripped, e.g., during manual insertion of the suppository into the body cavity.

### RELATED APPLICATIONS

This application claims priority to U.S. Provisional Patent Application No. 62/928,679, filed on Oct. 31, 2019, which is herein incorporated by reference in its entirety.

### FIELD OF THE INVENTION

The present invention relates to suppository capsules, and specifically to a softgel suppository capsule, methods of preparations thereof, and methods of use thereof.

### BACKGROUND OF THE INVENTION

Suppositories are typically shaped as bullets, tampons, ovals, ovoids, teardrop, torpedoes, and so on. Regardless of the shape, suppositories may be challenging to handle and/or administer. The direction for inserting the suppository may be unclear in some shapes. In some instances, certain shapes may easily fall from a patient's hands making handling and/or administration challenging. These challenges may be partially addressed by providing handling and/or administration instructions. Nevertheless, manufacturing suppositories that are user friendly as to the manner of handling and/or administration may be a beneficial addition to the existing suppository market.

### OBJECTS AND SUMMARY OF THE INVENTION

The present disclosure may be directed to a suppository comprising a suppository base. The suppository may have a length, measured along a horizontal axis, greater than a diameter, measured along a vertical axis. The suppository may have a proximal end and a distal end, along the horizontal axis. The proximal end of the suppository may be adapted for insertion into a body cavity of a subject. The distal end of the suppository may have at least one indentation.

The suppository may be a softgel suppository capsule with at least one indentation for an “easy grip” that would ease handling and/or administration of the suppository. The indented suppository shape described herein may be implemented with any suppository shape, such as, without limitations, an indented bullet, and indented oval, an indented ovoid, an indented teardrop, or an indented torpedo. The at least one indentation may be, without limitations, flat, concave, or perforated.

The suppositories described herein may comprise an active agent. In some embodiments, the suppository may have a core and shell structure and the active agent may be present in the core only, in the shell only, or in both—the core and the shell. In certain embodiments, the active agent may be dispersed throughout the suppository in its entirety. In certain embodiments, the active agent may not be dispersed in at least a portion of the suppository (e.g., at least in the distal portion of the suppository).

Certain embodiments of the instant disclosure may be directed to a method of preparing any of the indented suppository capsules described herein. The method may comprise encapsulating (e.g., via rotary die stamping) a liquid core comprising a suppository base in a shell (e.g., a gelatin shell). The method may also comprise dipping a solid core comprising a suppository base in a liquid shell composition (e.g., gelatin shell composition).

Certain embodiments of the instant disclosure may be directed to a method of treating a disease or a condition, such as, without limitations, pain, migraine, inflammation, fungal infection, bacterial infection, viral infection, vaginal pain, menopause, vulvar and vaginal atrophy, cancer, nausea, vomiting, or a combination thereof. The method may comprise inserting any of the indented suppositories described herein into a body cavity, such as, without limitations, the rectum, the vagina, or the urethra.

### BRIEF DESCRIPTION OF THE DRAWINGS

The above and other features of the present disclosure, their nature, and various advantages will become more apparent upon consideration of the following detailed description, taken in conjunction with the accompanying drawings, in which:

FIG. 1 is a perspective view of a suppository capsule in accordance with a first embodiment;

FIG. 2 is a top plan view of the first embodiment;

FIG. 3 is a bottom plan view of the first embodiment;

FIG. 4 is a left side elevation view of the first embodiment;

FIG. 5 is a right side elevation view of the first embodiment;

FIG. 6 is a back view of a distal end of the first embodiment;

FIG. 7 is a front view of a proximal end of the first embodiment;

FIG. 8 is a perspective view of a suppository capsule in accordance with a second embodiment;

FIG. 9 is a top plan view of the second embodiment;

FIG. 10 is a bottom plan view of the second embodiment;

FIG. 11 is a left side elevation view of the second embodiment;

FIG. 12 is a right side elevation view of the second embodiment;

FIG. 13 is a back view of a distal end of the second embodiment;

FIG. 14 is a front view of a proximal end of the second embodiment;

FIG. 15 is a perspective view of a suppository capsule in accordance with a third embodiment;

FIG. 16 is a top plan view of the third embodiment;

FIG. 17 is a bottom plan view of the third embodiment;

FIG. 18 is a left side elevation view the third embodiment;

FIG. 19 is a right side elevation view the third embodiment;

FIG. 20 is a back view of a distal end of the third embodiment;

FIG. 21 is a front view of a proximal end of the third embodiment;

FIG. 22 is a perspective view of a suppository capsule in accordance with a fourth embodiment;

FIG. 23 is a top plan view of the fourth embodiment;

FIG. 24 is a bottom plan view of the fourth embodiment;

FIG. 25 is a left side elevation view of the fourth embodiment;

FIG. 26 is a right side elevation view of the fourth embodiment;

FIG. 27 is a back view of a distal end of the fourth embodiment;

FIG. 28 is a front view of a proximal end of the fourth embodiment;

FIG. 29 is a perspective view of a suppository capsule in accordance with a fifth embodiment;

FIG. 30 is a top plan view of the fifth embodiment;

FIG. 31 is a bottom plan view of the fifth embodiment;

FIG. 32 is a left side elevation view of the fifth embodiment;

FIG. 33 is a right side elevation view of the fifth embodiment;

FIG. 34 is a back view of a distal end of the fifth embodiment; and

FIG. 35 is a front view of a proximal end of the fifth embodiment.

### DEFINITIONS

As used herein, the terms “active agent” “pharmaceutically active ingredient” refer to any material that is intended to produce a therapeutic, prophylactic, or other intended effect and is used in the diagnosis, cure, mitigation, treatment or prevention of a condition, whether or not approved by a government agency for that purpose. These terms with respect to specific agents include all pharmaceutically active agents, all pharmaceutically acceptable salts thereof, stereoisomers, crystalline forms, co-crystals, ether, esters, hydrates, solvates, and mixtures thereof, where the form is pharmaceutically active.

The terms “treatment of” and “treating” include the administration of an active agent(s) with the intent to lessen the severity of or prevent a condition.

The terms “prevention of” and “preventing” include the avoidance of the onset of a condition.

The term “condition” or “conditions” refers to those medical conditions that can be treated, mitigated or prevented by administration to a subject of an effective amount of an active agent. Exemplary non-limiting conditions that may benefit from suppository capsules described herein may include, without limitation, pain, migraine, inflammation, fungal infection, bacterial infection, viral infection, vaginal pain, menopause, vulvar and vaginal atrophy, cancer, nausea or vomiting.

The suppository capsules according to the disclosure may include various active agents and their pharmaceutically acceptable salts thereof. Pharmaceutically acceptable salts include, but are not limited to, inorganic acid salts such as hydrochloride, hydrobromide, sulfate, phosphate and the like; organic acid salts such as formate, acetate, trifluoroacetate, maleate, tartrate and the like; sulfonates such as methanesulfonate, benzenesulfonate, p-toluenesulfonate, and the like; amino acid salts such as arginate, asparginate, glutamate and the like, and metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like.

An “effective amount” or a “therapeutically effective amount” refers to the amount of an active agent in a pharmaceutical composition (such as a suppository capsule) that is sufficient to produce a beneficial or desired effect at a level that is readily detectable by a method commonly used for detection of such an effect. In some embodiments, such an effect results in a change of at least 10% from the value of a basal level where the active agent is not administered. In other embodiments, the change is at least 20%, 50%, 80%, or an even higher percentage from the basal level. As will be described below, the effective amount of an active agent may vary from subject to subject, depending on age, general condition of the subject, the severity of the condition being treated, and the particular active agent administered and the like. An appropriate “effective” amount in any individual case may be determined by one of ordinary skill in the art by reference to the pertinent texts and literature and/or by using routine experimentation.

As used herein, “shell” or “shell composition” refers to a constituent of a softgel suppository capsule which encapsulates a suppository base.

As used herein, “suppository base,” refers to a pharmaceutically acceptable material that is suitable to be used as a suppository delivery system.

As used herein, a “patient” refers to a subject, particularly a human (but could also encompass a non-human), who has presented a clinical manifestation of a particular symptom or symptoms suggesting the need for treatment, who is treated prophylactically for a condition, or who has been diagnosed with a condition to be treated.

The term “subject” encompasses the definition of the term “patient” and does not exclude individuals who are otherwise healthy.

The terms “suppository,” “suppository capsule,” and “dosage form” may all be used interchangeably throughout the description.

As used herein, the singular forms “a,” “an,” and “the” include plural references unless the context clearly indicates otherwise. Thus, for example, reference to “an active agent” includes a single active agent as well as a mixture of two or more different active agents; and reference to a “condition” includes a single condition as well as a mixture of two or more different conditions, and the like.

As used herein, the term “about” in connection with a measured quantity, refers to the normal variations in that measured quantity, as expected by one of ordinary skill in the art in making the measurement and exercising a level of care commensurate with the objective of measurement and the precision of the measuring equipment. In certain embodiments, the term “about” includes the recited number ±10%, such that “about 10” would include from 9 to 11.

The term “at least about” in connection with a measured quantity refers to the normal variations in the measured quantity, as expected by one of ordinary skill in the art in making the measurement and exercising a level of care commensurate with the objective of measurement and precisions of the measuring equipment and any quantities higher than that. In certain embodiments, the term “at least about” includes the recited number minus 10% and any quantity that is higher such that “at least about 10” would include 9 and anything greater than 9. This term can also be expressed as “about 10 or more.” Similarly, the term “less than about” typically includes the recited number plus 10% and any quantity that is lower such that “less than about 10” would include 11 and anything less than 11. This term can also be expressed as “about 10 or less.”

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context.

The use of any and all examples, or exemplary language (e.g., “such as”) provided herein, is intended merely to illuminate certain materials and methods and does not pose a limitation on scope. No language in the specification should be construed as indicating any non-claimed element as essential to the practice of the disclosed materials and methods.

### DETAILED DESCRIPTION

The present disclosure may be directed to a suppository dosage form designed with at least one indentation that allows the dosage form to be gripped and/or administered with one or two fingers. The indentation may also assist in the administration of the dosage form, for instance, by making the direction for inserting the suppository obvious. The indentation may be designed ergonomically to allow for a comfortable grip and efficient insertion of the suppository. The indentation may also be shaped to prevent expelling or slippage of the suppository from a body cavity.

The indentation may be applied to existing suppository shapes (such as bullets, tampons, ovals, ovoids, teardrops, torpedoes, and so on) by modifying one portion (e.g., one end) of the dosage form shape. In some embodiments, the grip section of suppository capsules described herein may have at least a portion of the active agent residing within it. In other embodiments, the grip section of suppository capsules described herein may be free of active agent.

Exemplary indented suppository capsules are described in more detail below with reference to the figures.

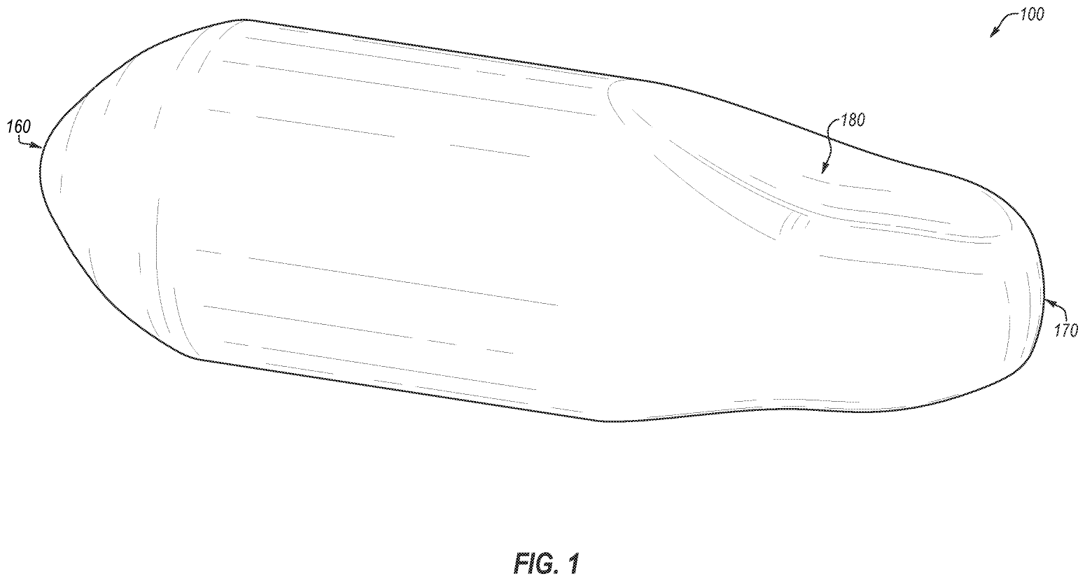

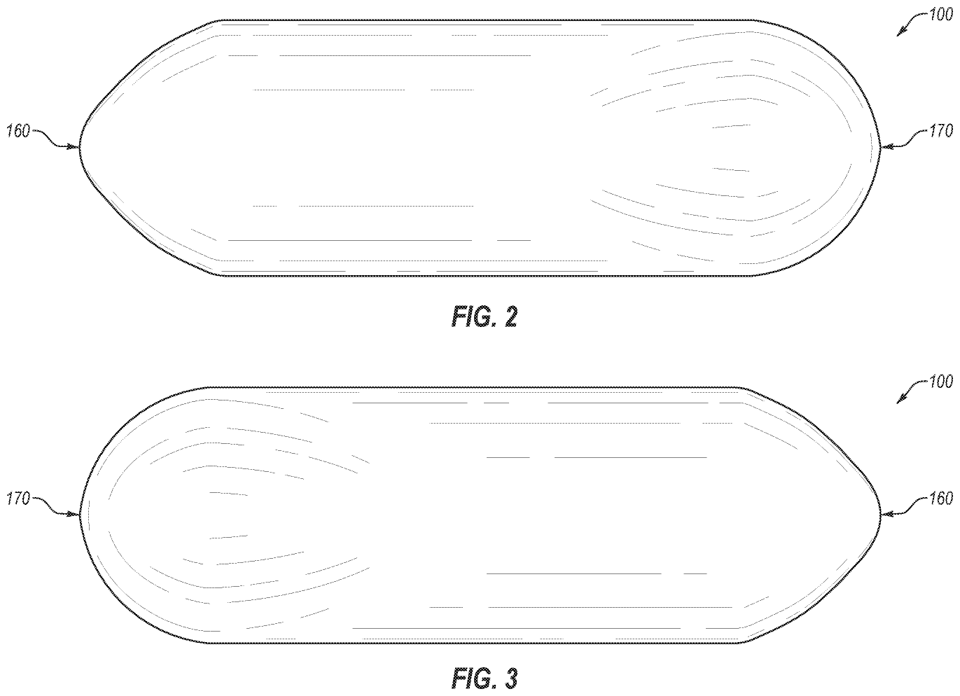

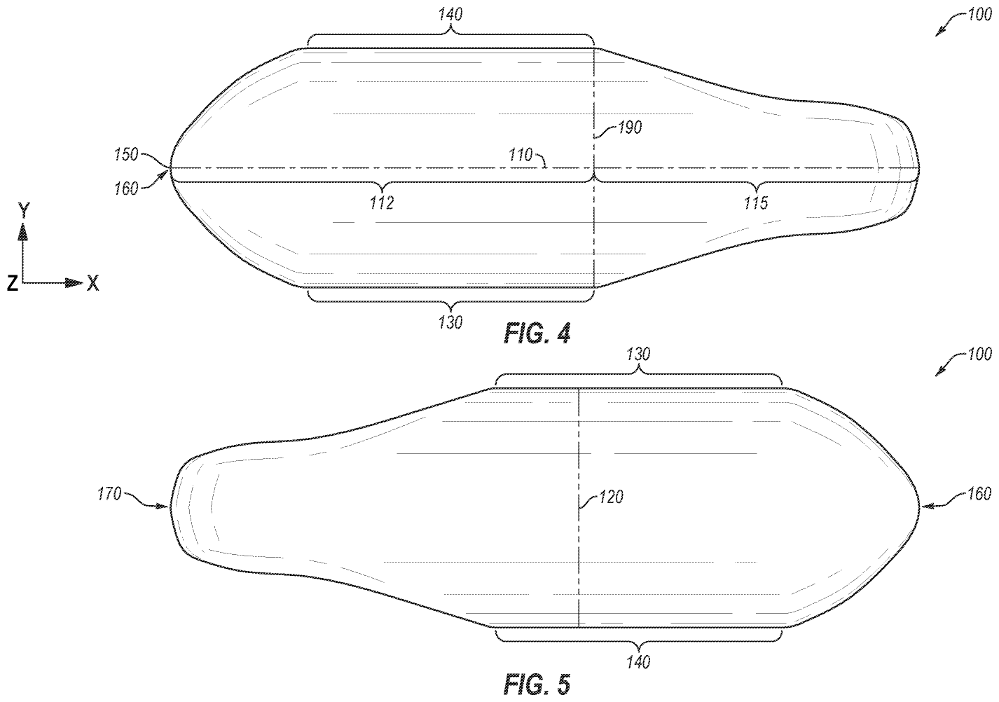

FIG. 1 depicts a perspective view of a suppository capsule in accordance with a first embodiment. FIG. 2 is a top plan view of the suppository according to the embodiment of FIG. 1. FIG. 3 is a bottom plan view of the suppository according to the embodiment of FIG. 1. FIG. 4 is a left side elevation view of the according to the embodiment of FIG. 1. FIG. 5 is a right side elevation view of the according to the embodiment of FIG. 1. As seen in FIGS. 1-5, the suppository capsule 100 may have an indented bullet shape, such that the proximal end 160 has a conventional bullet shape and the distal end 170 has at least one indentation. It should be understood that the bullet shape depicted in FIGS. 1-5 (of a cylindrical portion ending with a dome or cone on the proximal end 160 of the suppository) is merely exemplary and non-limiting. For instance, the dome or cone ending may be more rounded or more pointed than depicted in the embodiment. The cylindrical portion of the bullet shaped proximal end may be a cylinder with a constant diameter throughout the length of the cylinder as shown in FIGS. 1-5. Alternatively the diameter or width of the cylinder may widen or become narrower throughout the length of the suppository (as shown in the indented teardrop embodiments described hereinbelow or as shown in the indented oval embodiment described hereinbelow).

In embodiments, the direction of insertion of the suppositories described herein into the body cavity may be readily ascertained due to the distinct shapes of the proximal end and the distal end of the suppository. The proximal end (e.g., 160) with the conventional suppository shape is designed to be first inserted into the body cavity and the distal end (e.g., 170) is designed to be gripped so as to be manually inserted into the body cavity.

As seen in FIG. 4 and FIG. 5, the suppository capsule 100 may have a length 110, measured along a horizontal axis X, and a width 120, measured along a vertical axis Y. Horizontal axis X and vertical axis Y are shown in FIGS. 4 and 5. In the embodiment shown in FIGS. 4 and 5, the width 120 also corresponds to the diameter of the suppository capsule on the cylindrical portion of the bullet shaped end of the suppository capsule. The length (e.g., 110) of the suppository is defined as the longest portion of the suppository, as measured along its horizontal axis X. The width (e.g., 120) of the suppository is defined as the widest portion of the suppository, as measured along its vertical axis Y. For the first embodiment, the width of the suppository is shown in FIGS. 4 and 5 as numeral 120. In the first embodiment, the width of the suppository 120 also corresponds to the diameter of the cylindrical portion of the bullet shaped proximal end.

The length of the suppository capsule, as measured along its horizontal axis X, may vary at different locations along the vertical axis Y depending on the shape of the suppository capsule. In certain embodiments, the center of the suppository, along the vertical Y axis, is the longest horizontal portion or dimension of the suppository. Hence, in certain embodiments, the length of the suppository is the length of the center of the suppository, along the vertical Y axis. In certain embodiments, the horizontal length of the suppository tapers off (or shortens) when moving away from the center of the suppository to the edges of the suppository, along the vertical Y axis. For instance, FIGS. 1-5 depict an indented bullet shaped suppository capsule according to a first embodiment. In such embodiment, the horizontal length of the suppository capsule may be shortest on the edges 130 and 140 (edges of the suppository capsule along the vertical axis Y, as viewed from the left side elevation view shown in FIG. 4 and from the right side elevation view shown in FIG. 5) and may gradually increase until longest horizontal length 110 is achieved in the center 150 of the suppository capsule (the center of the suppository capsule along vertical axis Y, as shown in FIGS. 4 and 5).

In certain embodiments, the horizontal length 110 of the suppository capsule may range from about 10 mm to about 50 mm, from about 12 mm to about 45 mm, from about 15 mm to about 40 mm, or from about 20 mm to about 35 mm. These dimensions are merely exemplary and should not be construed as limiting.

The width of the suppository, as measured along its vertical axis Y, may also vary at different locations along the horizontal axis X depending on the shape of the suppository capsule. For instance, the suppository of the first embodiment, may have one width on the proximal end 160 of the suppository and another width on the distal end 170 of the suppository. The suppository capsule may have proximal end 160 on one end of horizontal axis X and a distal end 170 on an opposite end of horizontal axis X. Proximal end 160 may be adapted for insertion into a body cavity. For instance, proximal end 160 may have a conventional suppository shape such as, without limitations, bullet, oval, ovoid, teardrop, torpedo, and so on. Distal end 170 may have at least one indentation 180 that may be adapted for gripping the suppository capsule for easy handling and/or administration.

In embodiments, the length 110 of the suppository capsule is composed of the length of the portion of the suppository having a conventional suppository shape (i.e., the length of the bullet shape) plus the length of the portion of the suppository having the indentation. The length of the portion of the suppository having the conventional suppository shape is shown in FIGS. 4 and 5 as length 112. The length of the portion of the suppository having at least one indentation is shown in FIGS. 4 and 5 as length 115. Length 112 together with length 115 may add up to length 110. Length 112 (i.e., length of the conventional suppository shape portion) and length 115 (of the indented portion) may be separated by boundary line 190.

In certain embodiments, the ratio of length 112 to length 115 may range from about 10:1 to about 1:10, from about 8:1 to about 1:8, from about 5:1 to about 1:5, from about 3:1 to about 1:3, from about 2:1 to about 1:2, from about 10:1 to about 1:1, from about 8:1 to about 1:1, from about 5:1 to about 1:1, from about 3:1 to about 1:1, from about 2:1 to about 1:1, from about 1:1 to about 1:2, from about 1:1 to about 1:3, from about 1:1 to about 1:5, from about 1:1 to about 1:8, or from about 1:1 to about 1:10. These dimensions are merely illustrative and should not be construed as limiting.

In some embodiments, the boundary line 190 between the proximal end 160 and the distal end 170 may be the widest portion of the suppository so as to minimize the likelihood of the suppository from getting expelled from the body cavity. In some embodiments, the widest portion of the suppository may be along length 112 (i.e. on the portion of the suppository having a conventional suppository shape). In some embodiments, the widest portion of the suppository may be along length 115 (i.e., on the portion of the suppository having the at least one indentation).

The width (e.g., 120) of the suppository capsule may range from about 1 mm to about 30 mm, from about 2 mm to about 25 mm, from about 3 mm to about 20 mm, or from about 4 mm to about 15 mm. These dimensions are merely illustrative and should not be construed as limiting.

In certain embodiments, the at least one indentation may be flat. In other embodiments, the at least one indentation may be concave (i.e., may curve inward) so as to be ergonomically adapted to be gripped by at least one finger. In yet other embodiments, the at least one indentation may be perforated and may optionally have a hole (e.g., a donut hole). In certain embodiments, the at least one indentation may be adapted to be pinched by two fingers or by a device for easy grip and insertion into a body cavity.

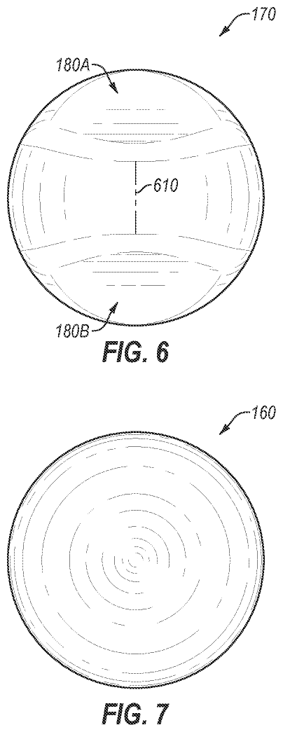

In an embodiment, distal end 170 may have two indentations 180A and 180B as shown in the back view (FIG. 6) of the suppository capsule according to the embodiment of FIGS. 1-5. In such an embodiment, each indentation of the two indentations may be on opposing sides of the distal end. The shortest distance between the boundaries of the two indentations may be designated by numeral 610 in FIG. 6 and may range from 0 mm to about 30 mm, from about 1 mm to about 25 mm, from about 2 mm to about 20 mm, from about 3 mm to about 15 mm, or any number of range in between. These dimensions are merely illustrative and should not be construed as limiting.

In another embodiment, the distal end of the suppository may have one indentation, three indentations, four indentations, five indentations, and so on. In certain embodiments, when two or more indentations are present, the indentations may be evenly spaced around the circumference of the distal end of the suppository. In certain embodiments, when two or more indentations are present, the indentations may not be evenly spaced around the circumference of the distal end of the suppository.

FIG. 6 is a back view of a distal end 170 of the first embodiment of a suppository capsule according to FIGS. 1-5. Two indentations 180A and 180B on opposing sides having a shortest distance 610 between their distal end boundaries are shown in FIG. 6. FIG. 7 is a front view of a proximal end 160 of the first embodiment depicting the front view of a bullet shape suppository having concentric circles to illustrate the dome shape associated with a bullet shaped proximal end.

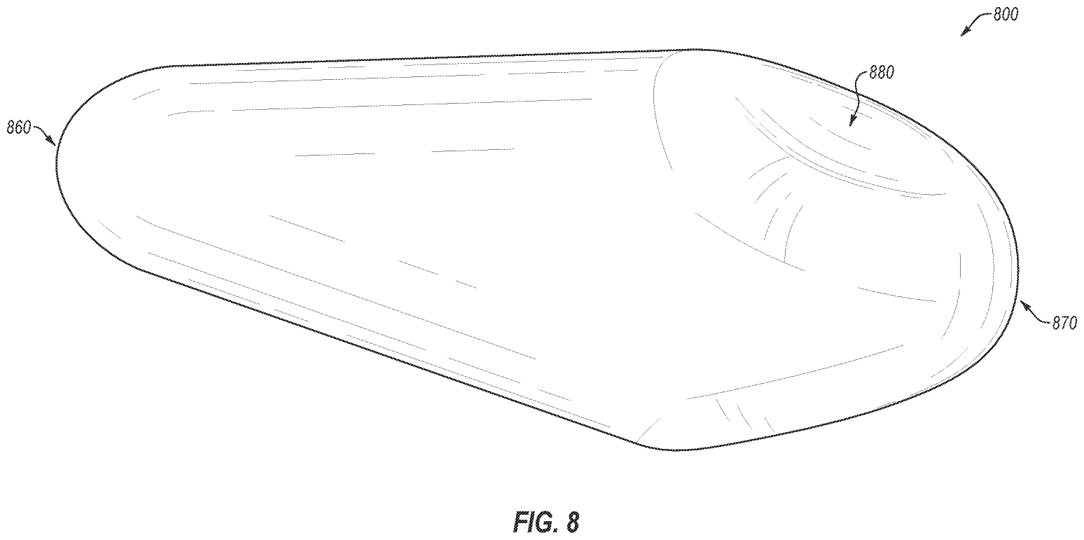

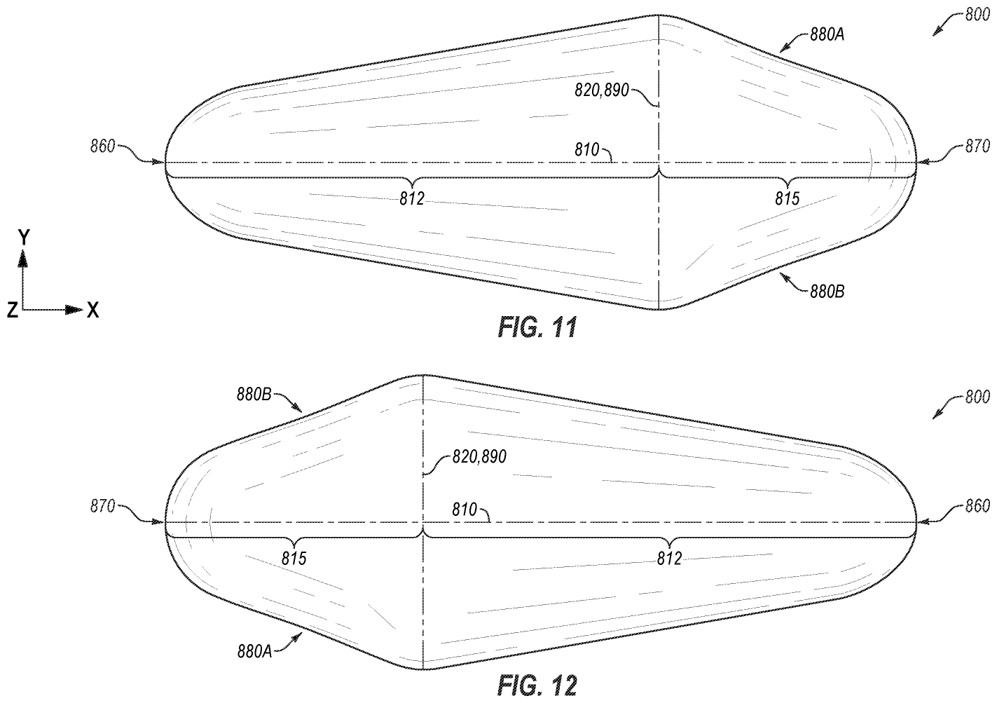

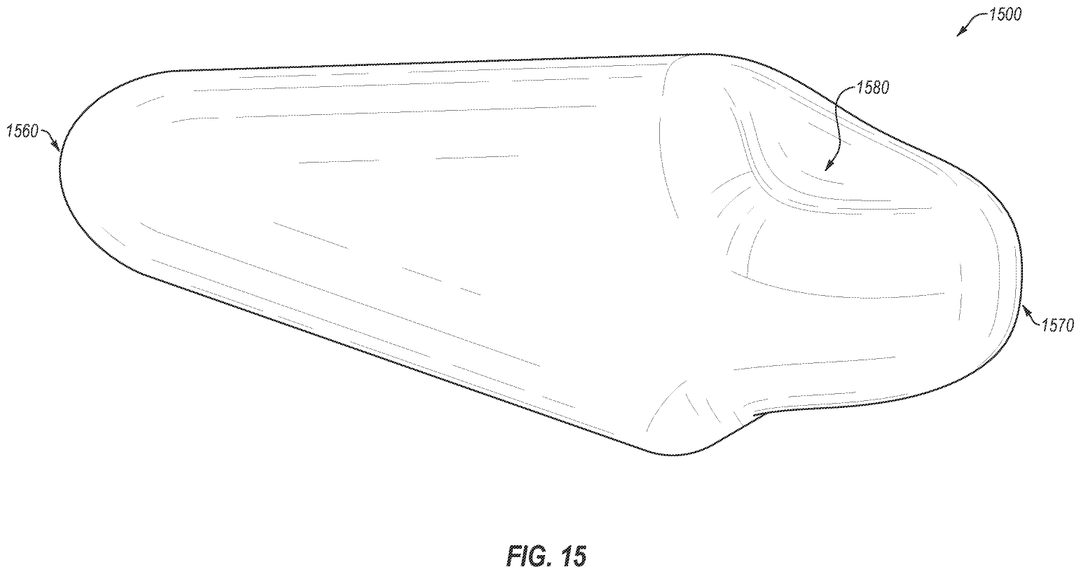

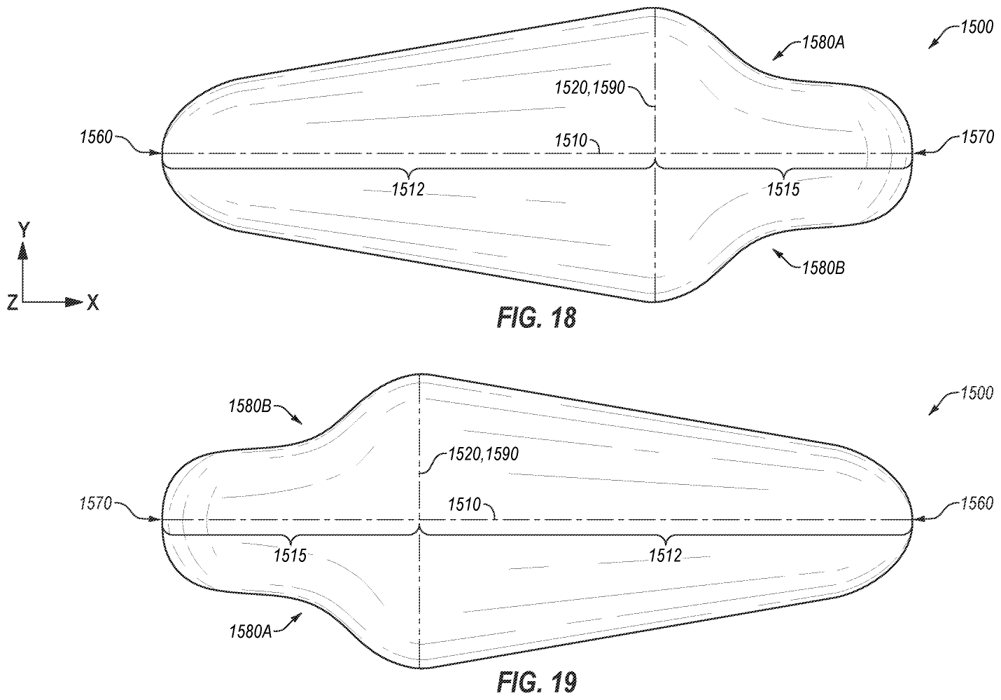

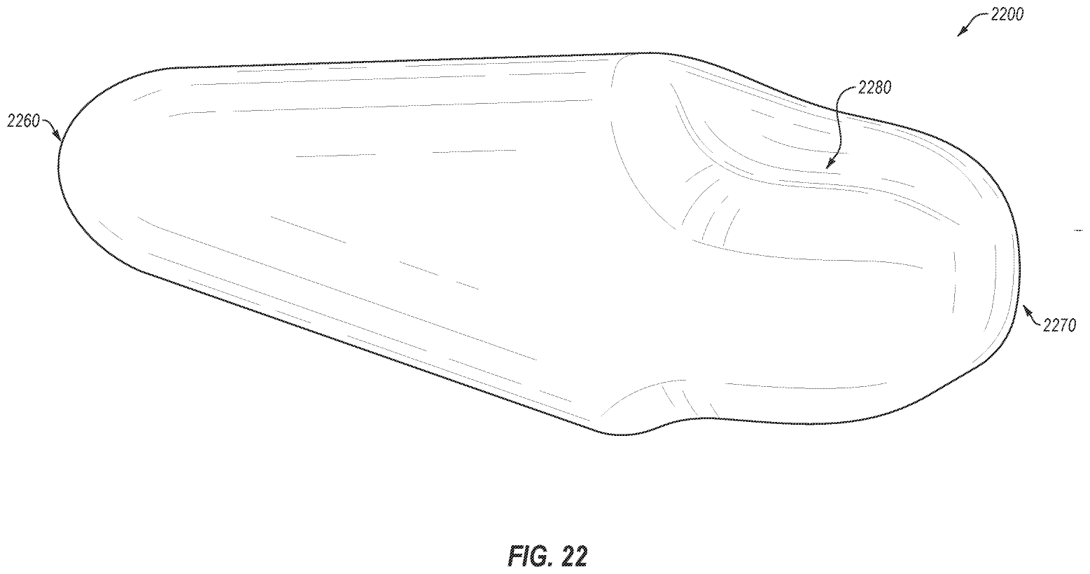

FIG. 8 is a perspective view of a suppository capsule 800 having an indented tear drop shape in accordance with a second embodiment. FIG. 15 is a perspective view of a suppository capsule 1500 having an indented tear drop shape in accordance with a third embodiment. FIG. 22 is a perspective view of a suppository capsule 2200 having an indented tear drop shape in accordance with a fourth embodiment. The suppository capsules 800, 1500, and 2200 may each have a length, measured along a horizontal axis X, and a width, measured along a vertical axis Y. The length of suppositories 800, 1500, and 2200, defined as the longest horizontal dimension of the suppository are shown as length 810 (FIGS. 11-12), 1510 (FIGS. 18-19), and 2210 (FIG. 25-26), respectively. The width of suppositories 800, 1500, and 2200, defined as the widest dimension of the suppository, as measured along its vertical axis Y, are shown as width 820 (FIGS. 11-12), width 1520 (FIGS. 18-19), and width 2220 (FIGS. 25-26), respectively.

The dimensions of the suppository capsule may vary depending on the shape of the suppository capsule. For instance, FIGS. 8 through 12, 15 through 19, and 22 through 26 depict an indented teardrop shaped suppository capsule according to various embodiments. These suppositories may have a pointy and elongated proximal end 860 (FIGS. 8-12), 1560 (FIGS. 15-19), and 2260 (FIGS. 22-26) having a gradually increasing width from the proximal end in the direction of the distal end 870 (FIGS. 8-12), 1570 (FIGS. 15-19), and 2270 (FIGS. 22-26) until the widest respective width 820, 1520, 2220 is achieved. Proximal ends 860, 1560, 2260 may be adapted for insertion into a body cavity while distal ends 870, 1570, 2270 may have at least one indentation 880 (FIG. 8), 1580 (FIG. 15), 2280 (FIG. 22) that may be adapted for gripping any of the suppository capsules for easy handling and/or administration.

The length 810, 1510, 2210 of the suppository capsules may range from about 10 mm to about 50 mm, from about 12 mm to about 45 mm, from about 15 mm to about 40 mm, or from about 20 mm to about 35 mm. These dimensions are merely illustrative and should not be construed as limiting.

In embodiments, the lengths 810, 1510, and 2210 of the suppository capsules of FIGS. 8-12, FIGS. 15-19, and FIGS. 22-26, respectively, is composed of the length of the portion of the suppository having a conventional suppository shape (i.e., the length of the teardrop shape) plus the length of the portion of the suppository having the indentation. The length of the portion of the suppository having a teardrop suppository shape is shown as length 812 (FIGS. 11-12), length 1512 (FIG. 18-19), length 2212 (FIG. 25-26). The length of the portion of the suppository having at least one indentation is shown as length 815 (FIG. 11-12), length 1515 (FIG. 18-19), length 2215 (FIG. 25-26). Length 812 together with length 815 may add up to length 810. Length 1512 together with length 1515 may add up to length 1510. Length 2212 together with length 2215 may add up to length 2210. Each of lengths 812, 1512, and 2212 (i.e., length of the conventional suppository shape portion) and each of respective lengths 815, 1515, and 2215 (of the indented portion) may be separated by a corresponding boundary line 890, 1590, and 2290.

The ratio of length 812 to length 815 (or of length 1512 to 1515, or of length 2212 to 2215) may range from about 10:1 to about 1:10, from about 8:1 to about 1:8, from about 5:1 to about 1:5, from about 3:1 to about 1:3, from about 2:1 to about 1:2, from about 10:1 to about 1:1, from about 8:1 to about 1:1, from about 5:1 to about 1:1, from about 3:1 to about 1:1, from about 2:1 to about 1:1, from about 1:1 to about 1:2, from about 1:1 to about 1:3, from about 1:1 to about 1:5, from about 1:1 to about 1:8, or from about 1:1 to about 1:10. These dimensions are merely illustrative and should not be construed as limiting.

In some embodiments, the boundary lines 890, 1590, and 2290 may be the widest portion of the suppository so as to minimize the likelihood of the suppository from getting expelled from the body cavity. In some embodiments, the widest portion of the suppository may be along lengths 812, 1512, or 2212 (i.e. on the portion of the suppository having a conventional suppository shape). In some embodiments, the widest portion of the suppository may be along length 815, 1515, or 2215 (i.e., on the portion of the suppository having the at least one indentation).

The width 820, 1520, 2220 of the suppository capsule may range from about 1 mm to about 30 mm, from about 2 mm to about 25 mm, from about 3 mm to about 20 mm, or from about 4 mm to about 15 mm. These dimensions are merely illustrative and should not be construed as limiting.

In certain embodiments, the at least one indentation in an indented teardrop suppository may be flat. In other embodiments, the at least one indentation in an indented teardrop suppository may be concave (i.e., may curve inward) so as to be ergonomically adapted to be gripped by at least one finger. In yet other embodiments, the at least one indentation in an indented teardrop suppository may be perforated and may optionally have a hole (e.g., a donut hole). In certain embodiments, the at least one indentation may be adapted to be pinched by two fingers or by a device for easy grip and insertion into a body cavity.

In an embodiment, distal end 870 may have two indentations 880A and 880B in FIGS. 11, 12, and 13. Similarly, distal end 1570 may have two indentations 1580A and 1580B in FIGS. 18, 19, and 20. Likewise, distal end 2270 may have two indentations 2280A and 2280B in FIGS. 25, 26, and 27. In such an embodiment, each indentation of the two indentations may be on opposing sides of the distal end. The shortest distance between the boundaries of the two indentations may be designated by numeral 861 (FIGS. 11, 12, and 13), 1561 (FIGS. 18, 19, and 20), or 2261 (FIGS. 25, 26, and 27). 861, 1561, or 2261 may range from 0 mm to about 30 mm, from about 1 mm to about 25 mm, from about 2 mm to about 20 mm, from about 3 mm to about 15 mm, or any number of range in between. These dimensions are merely illustrative and should not be construed as limiting.

In another embodiment, the distal end of an indented teardrop suppository may have one indentation, three indentations, four indentations, five indentations, and so on. In certain embodiments, when two or more indentations are present, the indentations may be evenly spaced around the circumference of the distal end of the suppository. In certain embodiments, when two or more indentations are present, the indentations may not be evenly spaced around the circumference of the distal end of the suppository.

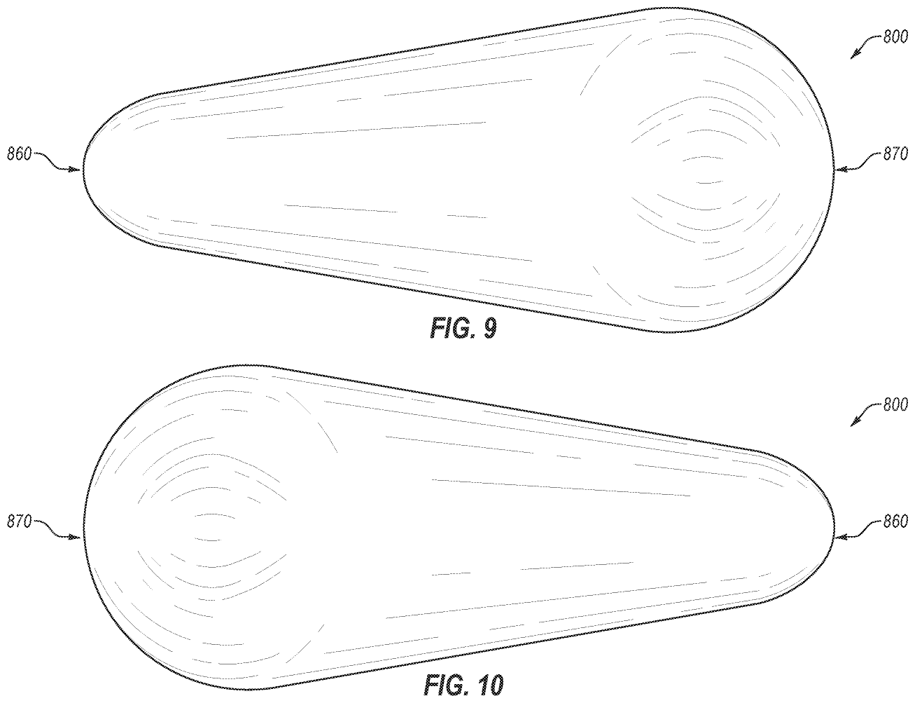

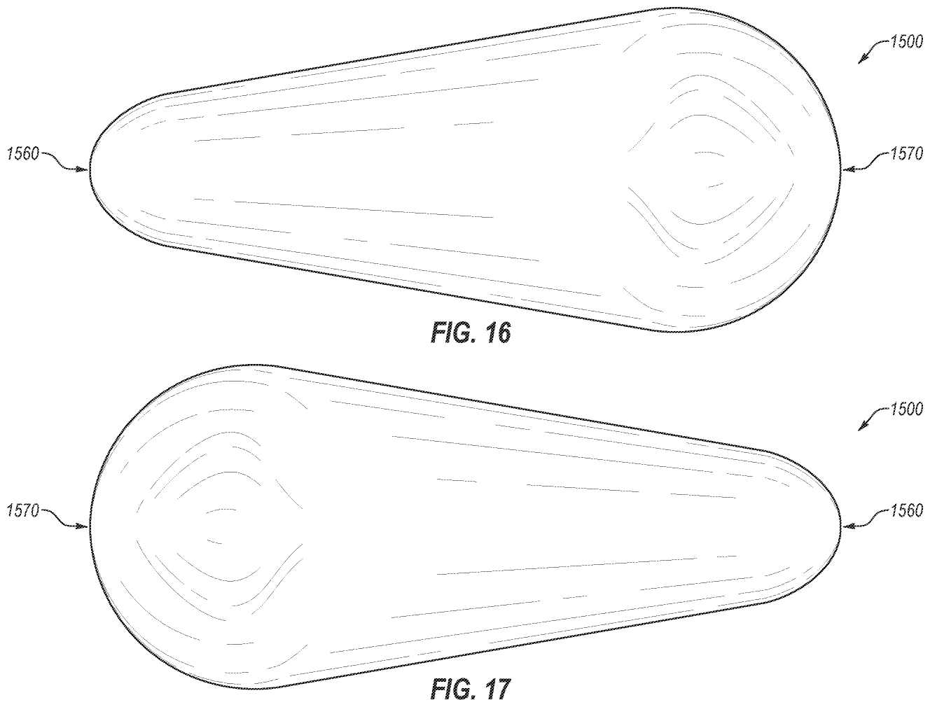

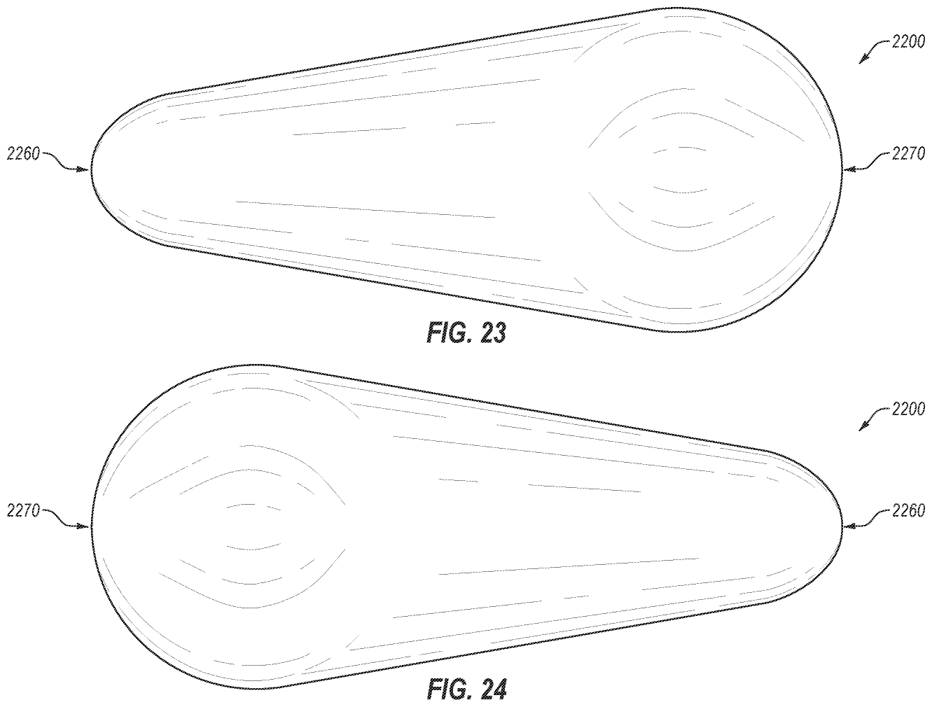

FIG. 9 is a top plan view of the second embodiment. FIG. 10 is a bottom plan view of the second embodiment. FIG. 16 is a top plan view of the third embodiment. FIG. 17 is a bottom plan view of the third embodiment. FIG. 23 is a top plan view of the fourth embodiment. FIG. 24 is a bottom plan view of the fourth embodiment.

As seen in FIGS. 9, 10, 16, 17, 23, and 24 the suppository may have various indented teardrop shapes, such as those shown in proximal ends 860 (FIGS. 9, 10), 1560 (FIG. 16, 17), or 2260 (FIGS. 23, 24). As seen in FIGS. 9, 10, 16, 17, 23, and 24 the suppository may also have indentations of various shapes, such as those shown in distal ends 870 (FIGS. 9, 10), 1570 (FIG. 16, 17), or 2270 (FIGS. 23, 24). The illustrated shapes for the proximal and distal ends should not be construed as limiting. A large number of suppository shapes can be attained due to the strength of the shell composition (e.g., the strength of a gelatin shell composition).

FIG. 11 is a left side elevation view of the second embodiment. FIG. 12 is a right side elevation view of the second embodiment. FIG. 18 is a left side elevation view of the third embodiment. FIG. 19 is a right side elevation view of the third embodiment. FIG. 25 is a left side elevation view of the fourth embodiment. FIG. 26 is a right side elevation view of the fourth embodiment. FIGS. 11, 12, 18, 19, 25, and 26, show the suppository's various possible indented teardrop shapes from different angles.

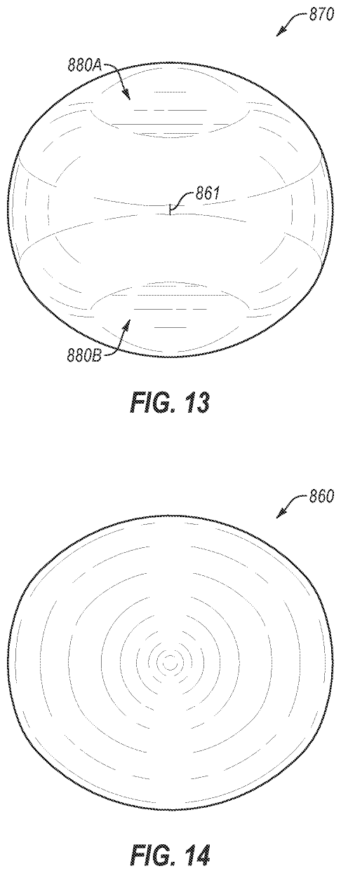

FIG. 13 is a back view of a distal end 870 of the second embodiment. Two indentations 880A and 880B on opposing sides having a shortest distance 861 between their distal end boundaries are shown in FIG. 13. FIG. 14 is a front view of a proximal end 860 of the second embodiment depicting the front view of a teardrop shaped suppository.

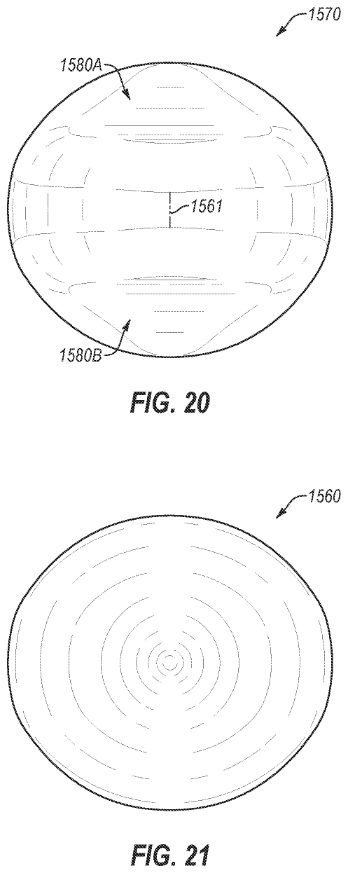

FIG. 20 is a back view of a distal end 1570 of the third embodiment. Two indentations 1580A and 1580B on opposing sides having a shortest distance 1561 between their distal end boundaries are shown in FIG. 20. FIG. 21 is a front view of a proximal end 1560 of the third embodiment depicting the front view of a teardrop shaped suppository.

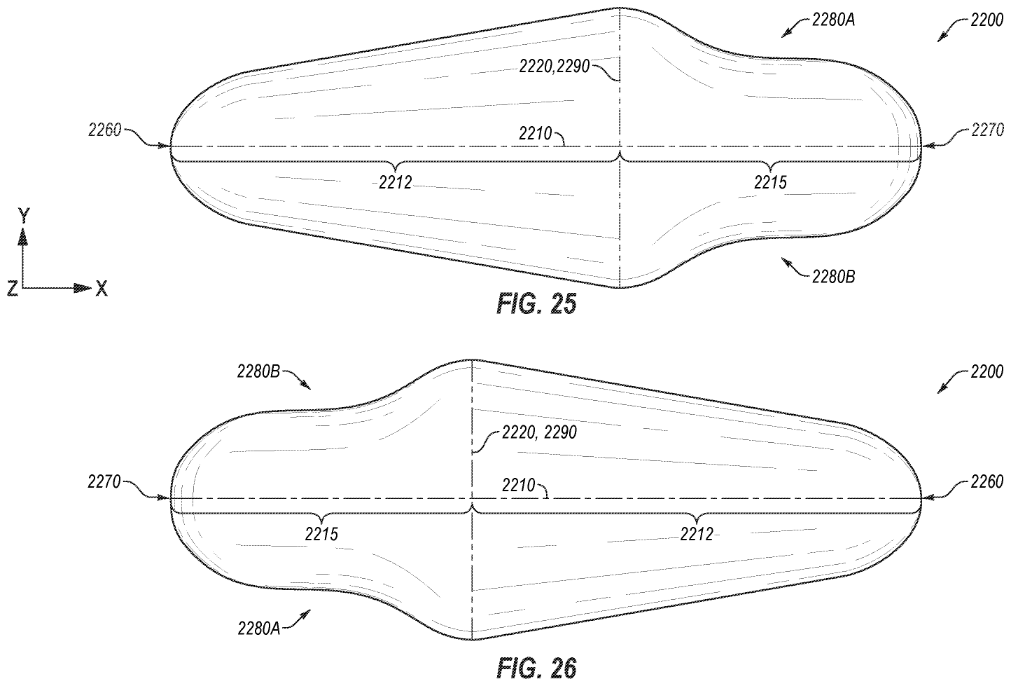

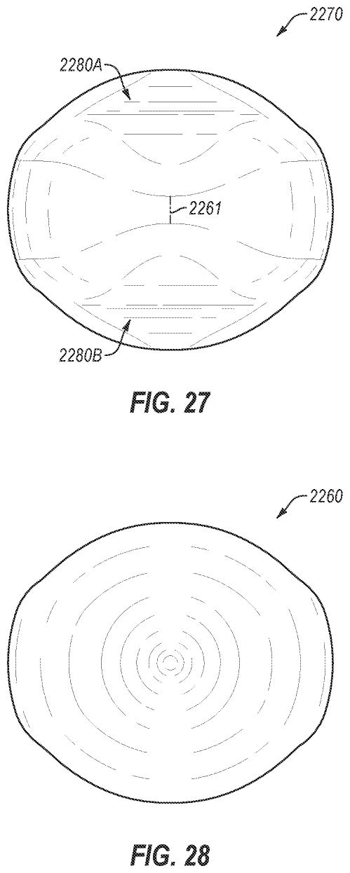

FIG. 27 is a back view of a distal end 2270 of the fourth embodiment. Two indentations 2280A and 2280B on opposing sides having a shortest distance 2261 between their distal end boundaries are shown in FIG. 27. FIG. 28 is a front view of a proximal end 2260 of the fourth embodiment depicting the front view of a teardrop shaped suppository.

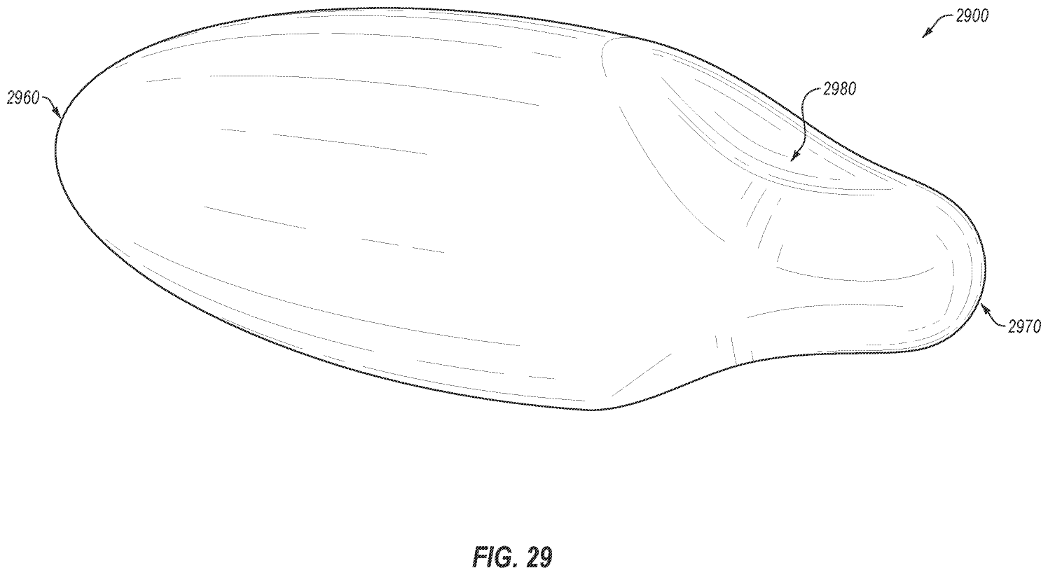

FIG. 29 is a perspective view of a suppository capsule 2900 having an indented oval shape in accordance with a fifth embodiment. The suppository capsules 2900 may have a length, measured along a horizontal axis X, and a width, measured along a vertical axis Y. The length of suppository 2900, defined as longest horizontal dimension of the suppository, as measured along its horizontal axis X, is depicted as length 2910. The widest dimension of the suppository 2900, as measured along its vertical axis Y, is depicted as width 2920.

The dimensions of the suppository capsule may vary depending on the shape of the suppository capsule. For instance, FIG. 29 depicts an indented oval shaped suppository capsule according to an embodiment. Proximal end 2960 may be adapted for insertion into a body cavity while distal end 2970 may have at least one indentation 2980 that may be adapted for gripping the suppository capsule for easy handling and/or administration.

The length 2910 of the suppository capsule may range from about 10 mm to about 50 mm, from about 12 mm to about 45 mm, from about 15 mm to about 40 mm, or from about 20 mm to about 35 mm. These dimensions are merely illustrative and should not be construed as limiting.

In embodiments, the length 2910 of the suppository capsule is composed of the length of the portion of the suppository having a conventional suppository shape (i.e., the length of the oval shaped end) plus the length of the portion of the suppository having the indentation. The length of the portion of the suppository having the oval suppository shape is depicted as length 2912. The length of the portion of the suppository having at least one indentation is depicted as length 2915. Length 2912 together with length 2915 may add up to length 2910. Length 2912 (i.e., length of the conventional suppository shape portion) and length 2915 (of the indented portion) may be separated by boundary line 2990.

The ratio of length 2912 to length 2915 may range from about 10:1 to about 1:10, from about 8:1 to about 1:8, from about 5:1 to about 1:5, from about 3:1 to about 1:3, from about 2:1 to about 1:2, from about 10:1 to about 1:1, from about 8:1 to about 1:1, from about 5:1 to about 1:1, from about 3:1 to about 1:1, from about 2:1 to about 1:1, from about 1:1 to about 1:2, from about 1:1 to about 1:3, from about 1:1 to about 1:5, from about 1:1 to about 1:8, or from about 1:1 to about 1:10. These dimensions are merely illustrative and should not be construed as limiting.

In some embodiments, the boundary line 2990 may be the widest portion of the suppository so as to minimize the likelihood of the suppository from getting expelled from the body cavity. In some embodiments, the widest portion of the suppository may be along length 2912 (i.e. on the portion of the suppository having a conventional suppository shape). In some embodiments, the widest portion of the suppository may be along length 2915 (i.e., on the portion of the suppository having the at least one indentation).

For instance, in FIG. 29 depicting an indented oval shaped suppository, according to a fifth embodiment, the widest portion of the suppository is along the oval (i.e., along length 2912) and may minimize the likelihood of the suppository getting expelled from the body cavity.

The diameter 2920 of the suppository capsule may range from about 1 mm to about 30 mm, from about 2 mm to about 25 mm, from about 3 mm to about 20 mm, or from about 4 mm to about 15 mm. These dimensions are merely illustrative and should not be construed as limiting.

In certain embodiments, the at least one indentation in an indented oval suppository may be flat. In other embodiments, the at least one indentation in an indented oval suppository may be concave (i.e., may curve inward) so as to be ergonomically adapted to be gripped by at least one finger. In yet other embodiments, the at least one indentation in an indented oval suppository may be perforated and may optionally have a hole (e.g., a donut hole). In certain embodiments, the at least one indentation may be adapted to be pinched by two fingers or by a device for easy grip and insertion into a body cavity.

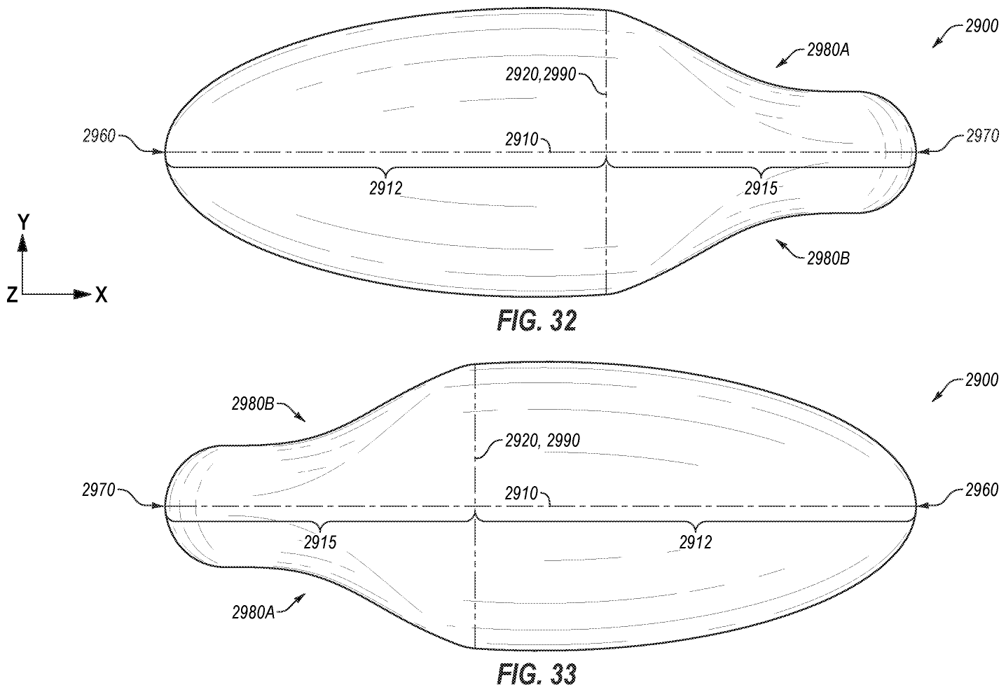

In an embodiment, distal end 2970 may have two indentations 2980A and 2980B in FIGS. 32, 33, and 34. In such an embodiment, each indentation of the two indentations may be on opposing sides of the distal end. The shortest distance between the boundaries of the two indentations may be designated by numeral 2961. Distance 2961 may range from 0 mm to about 30 mm, from about 1 mm to about 25 mm, from about 2 mm to about 20 mm, from about 3 mm to about 15 mm, or any number of range in between. These dimensions are merely illustrative and should not be construed as limiting.

In another embodiment, the distal end of an indented teardrop suppository may have one indentation, three indentations, four indentations, five indentations, and so on. In certain embodiments, when two or more indentations are present, the indentations may be evenly spaced around the circumference of the distal end of the suppository. In certain embodiments, when two or more indentations are present, the indentations may not be evenly spaced around the circumference of the distal end of the suppository.

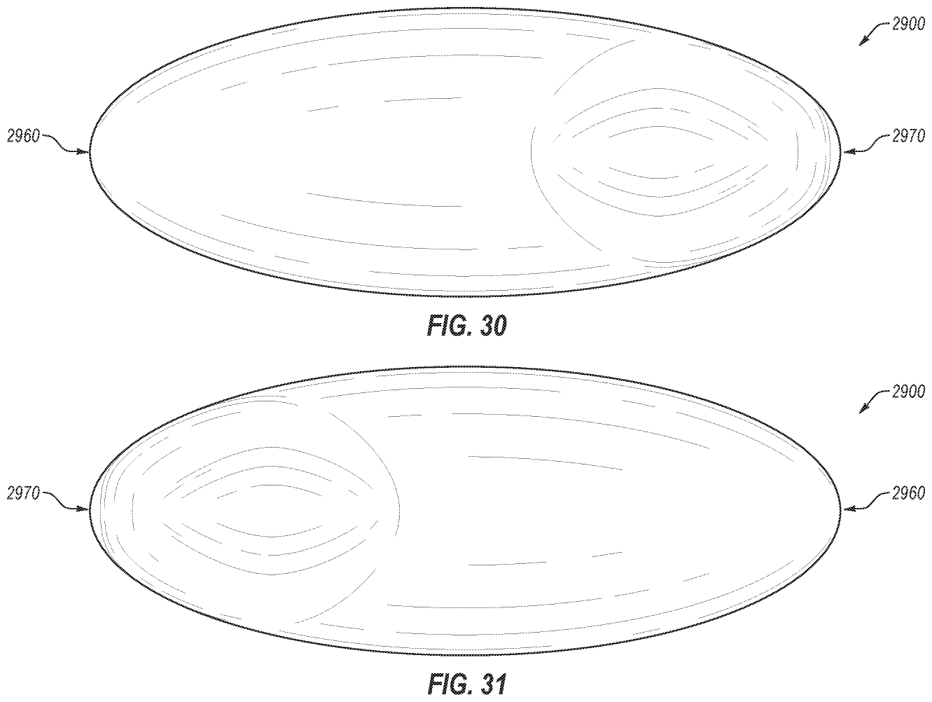

FIG. 30 is a top plan view of the fifth embodiment. FIG. 31 is a bottom plan view of the fifth embodiment. As seen in FIGS. 30 and 31 the suppository may have an indented oval shape, such that the proximal end 2960 has an oval shape and the distal end 2970 has at least one indentation.

FIG. 32 is a left side elevation view of the fifth embodiment. FIG. 33 is a right side elevation view of the fifth embodiment. FIGS. 32 and 33 also show the suppository's indented oval shape from a different angle.

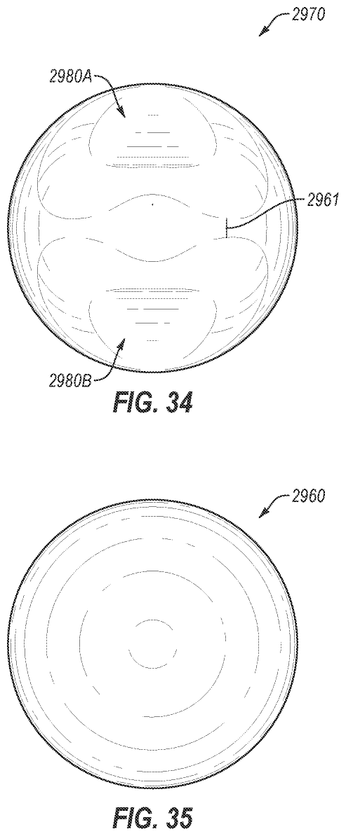

FIG. 34 is a back view of a distal end 2970 of the fifth embodiment. Two indentations 2980A and 2980B on opposing sides having a shortest distance 2961 between their distal end boundaries are shown in FIG. 34. FIG. 35 is a front view of a proximal end 2960 of the fifth embodiment depicting the front view of an oval shaped suppository.

Although the suppository shapes depicted in the figures were indented bullet shape, three various indented teardrop shapes, and an indented oval shape, other indented suppository capsule shapes are also encompassed herein. For instance, the instant disclosure may encompass shapes such as, without limitations, indented bullet, indented tampon, indented oval (e.g., indented long oval or indented round oval), indented ovoid, indented teardrop indented torpedoes, and so on.

The suppository capsule may comprise a suppository base that may be robust enough to permit construction of the indented suppository shapes described herein. The suppository base may comprise, without limitations, one or more of cocoa butter, lauric oil, beef tallow, hard fat, theobroma oil, glycerides of fatty acids, glycerol-gelatin bases, or a combination thereof. The suppository base may be in a liquid form or in a solid form. In an embodiment, the suppository base may have a melting point at the body core temperature (e.g., from about 30° C. to about 45° C., from about 33° C. to about 42° C., from about 35° C. to about 40° C., or about 37° C.) such that the suppository base may be in a solid form at ambient temperature outside the body and may melt into a liquid form upon insertion of the suppository into a body cavity.

The suppository may enclose a suppository base (e.g., in a core and shell configuration). The suppository base may comprise an active agent. In certain embodiments, the active agent may be dispersed throughout the suppository base in its entirety. In other embodiments, the active agent may be dispersed in certain portions of the suppository base and may not be dispersed throughout the suppository base in its entirety. For instance, in some embodiments, the active agent may not be dispersed throughout the suppository base in at least a portion of the distal end of the suppository capsule, such as where there is at least one indentation for easy grip of the suppository.

### Core and Shell Formulation

In certain embodiments, the suppository capsule may have a core and a shell structure and the active agent may be contained in the core only, in the shell only, or in both—the core and the shell. In one embodiment, the active agent may be contained in the core only. In one embodiment, the active agent may be contained in the shell only. In one embodiment, the active agent may be containing in the core and in the shell. Regardless of where the active agent is contained (core, shell, or both) it may be dispersed throughout or may not be dispersed in at least one portion (e.g., in a distal portion of the core, shell, or both).

In an embodiment, the distal portion of the suppository may be free of active agent such that the at least one indentation may be flat and may form a flat “grip area.” In another embodiment, the active agent may be dispersed throughout the suppository (including in the at least one indentation in the distal end of the suppository) such that the at least one indentation may form a concave “grip area.” These embodiments are merely exemplary as any “grip area” or indentation shape may be designed to accommodate an active agent that is dispersed throughout the suppository or an active agent that is dispersed only in a portion of the suppository (e.g., when the distal end of the suppository is free of active agent).

In certain embodiments, the shell may be robust enough to permit construction of at least one indentation (for an “easy grip”) in the distal portion and a conventional suppository shape in the proximal portion of the suppository. In some embodiments, the core (or the suppository base) may be robust enough to permit construction of at least one indentation (for an “easy grip”) in the distal portion and a conventional suppository shape in the proximal portion of the suppository. In some embodiments, the shell may comprise, without limitations, at least one of gelatin, starch, modified starch, carrageenan, alginate, a biodegradable polymer, or a combination thereof.

In certain embodiments, the shell composition comprises gelatin. The gelatin may comprise, without limitations, Type A gelatin, Type B gelatin, or mixtures thereof. The gelatin may comprise, without limitations, fish gelatin, hide gelatin, bone gelatin, or mixtures thereof. The gelatin may be present in the shell in an amount ranging from about 40% w/w to about 80% w/w, from about 45% w/w to about 75% w/w, or from about 50% w/w to about 70% w/w, based on total weight of the shell composition.

In certain embodiments, the shell composition comprises carrageenan. The carrageenan in the shell composition may be kappa-carrageenan, iota-carrageenan, lambda-carrageenan and mixtures thereof. According to one embodiment, the carrageenan is kappa-carrageenan. According to another embodiment, the carrageenan is iota-carrageenan. In an embodiment, the amount of the carrageenan in the shell composition is about 2% w/w to about 10% w/w, from about 2% w/w to about 8% w/w, or from about 2% w/w to about 5% w/w, based on total weight of the shell composition.

In certain embodiments, the shell composition comprises a biodegradable polymer. The biodegradable polymer may comprise, without limitations, polylactic acid (PLA), poly(ε-caprolactone) (PCL), poly(lactide-co-glycolic acid) (PLGA), or a combination thereof.

In certain embodiments, the shell may comprise a plasticizer, such as, without limitations, glycerol, sorbitol, or mixtures thereof. Other suitable plasticizers may include, but not be limited to, sugar alcohol plasticizer such as isomalt, maltitol, xylitol, erythritol, adonitol, dulcitol, pentaerythritol, or mannitol; or polyol plasticizer such as diglycerin, ethylene glycol, diethylene glycol, triethyleneglycol, tetraethylene glycol, dipropylene glycol, a polyethylene glycol up to 10,000 MW, neopentyl glycol, propylene glycol, 1,3-propanediol, 2-methyl-1,3-propanediol, trimethylolpropane, a polyether polyol, ethanol amines; and mixtures thereof. Other exemplary plasticizers may also include, without limitations, low molecular weight polymers, oligomers, copolymers, oils, small organic molecules, low molecular weight polyols having aliphatic hydroxyls, ester-type plasticizers, glycol ethers, poly(propylene glycol), multi-block polymers, single block polymers, citrate ester-type plasticizers, and triacetin. Such plasticizers may include 1,2-butylene glycol, 2,3-butylene glycol, styrene glycol, monopropylene glycol monoisopropyl ether, propylene glycol monoethyl ether, ethylene glycol monoethyl ether, diethylene glycol monoethyl ether, sorbitol lactate, ethyl lactate, butyl lactate, ethyl glycolate, dibutyl sebacate, acetyltributylcitrate, triethyl citrate, glyceryl monostearate, polysorbate 80, acetyl triethyl citrate, tributyl citrate and allyl glycolate, and mixtures thereof. The plasticizer may be present in the shell in an amount ranging from about 15% w/w to about 40% w/w, from about 20% w/w to about 35% w/w, or from about 25% w/w to about 30% w/w, based on total weight of the shell composition.

In one embodiment, the suppository may have a core and a shell structure such that the core comprises the suppository base. In certain embodiments, the core may be solid at ambient temperature and may melt in core body temperature (e.g., from about 30° C. to about 45° C., from about 33° C. to about 42° C., from about 35° C. to about 40° C., or about 37° C.). In other embodiments, the core may be liquid at ambient temperature.

### Active Agents

Suitable active agents that may be contained in the suppository capsules described herein may include, without limitations, an analgesic, anti-migraine, anti-inflammatory, anti-fungal, antibiotic, anti-viral, hormone, chemotherapeutic, anti-nausea, anti-emetic, or a combination thereof.

Exemplary analgesic active agents may include, without limitations, acetaminophen or an opioid. Examplary opioids may include, without limitations, alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, etorphine, dihydroetorphine, fentanyl and derivatives, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, nalorphine, nalbuphene, normorphine, norpipanone, opium, oxycodone, oxymorphone, papaveretum, pentazocine, phenadoxone, phenomorphan, phenazocine, phenoperidine, piminodine, piritramide, propheptazine, promedol, properidine, propoxyphene, sufentanil, tilidine, tramadol, pharmaceutically acceptable salts thereof, and combinations thereof.

Exemplary anti-migraine active agents may include, without limitations, ergotamine, dihydroergotamine, ergostine, butalbital, phenobarbital, sumatriptan, naratriptan, razatriptan, zolmitriptan, almotriptan, eletriptan, valproic acid, gabapentin, topiramate, divalproex, pharmaceutically acceptable salts thereof or mixtures thereof.

Exemplary anti-inflammatory active agents may include, without limitations, ibuprofen, fenoprofen, naproxen, sulindac, diclofenac, piroxicam, ketoprofen, diflunisal, nabumetone, etodolac, oxaprozin, indomethacin, pharmaceutically acceptable salts thereof or mixtures thereof.

Exemplary anti-fungal agents include, without limitations, abafungin, albaconazole, amorolfin, amphotericin b, anidulafungin, bifonazole, butenafine, butoconazole, candicidin, caspofungin, ciclopirox, clotrimazole, econazole, fenticonazole, filipin, fluconazole, flucytosine, griseofulvin, haloprogin, hamycin, isavuconazole, isoconazole, itraconazole, ketoconazole, micafungin, miconazole, naftifine, natamycin, nystatin, omoconazole, oxiconazole, polygodial, posaconazole, ravuconazole, rimocidin, sertaconazole, sulconazole, terbinafine, terconazole, tioconazole, tolnaftate, undecylenic acid, voriconazole, pharmaceutically acceptable salts thereof or mixtures thereof.

Exemplary antibiotic active agents may include, without limitations, mitomycin, ciprofloxacin, norfloxacin, ofloxacin, methanaminc, nitrofurantoin, ampicillin, amoxicillin, nafcillin, trimethoprim, sulfonamides trimethoprim-sulfamethoxazole, erythromycin, doxycycline, metronidazole, tetracycline, kanamycin, penicillins, cephalosporins, aminoglycosides, pharmaceutically acceptable salts thereof or mixtures thereof.

Exemplary anti-viral active agents may include, without limitations, abacavir, acyclovir, adefovir, amprenavir, atazanavir, atazanavir, cidofovir, darunavir, delavirdine, didavines, didanosine, docosanol, efavirenz, erbitegra, emtricitabine, entravirin, femciclovir, foscarnet, homivirsen, ganciclovir, Indinavir, idoxuridine, laminivazine, nelfinavir, nevirapine, penciclovir, raltegravir, rilpivirine, rilupavirin, ritonavir, saquinavir, stavudine, tenofovir trifluridine, valacyclovir, valganciclovir, vidarabine, ivacitabine, tipranavir, zalcitabine, zidovudine, pharmaceutically acceptable salts thereof and mixtures thereof.

Exemplary hormone active agents include, without limitations, estradiol and pharmaceutically acceptable salts thereof. Additional hormone active agents may include, without limitations, progesterone and pharmaceutically acceptable salts thereof.

Exemplary anti-nausea active agents may include, without limitations, metoclopramide, prochlorperazine, domperidone, ondansetron, tropisetron, dolasetron, nabilone, dronabinol, levonantradol, aprepitant, cyclizine, promethazine, pharmaceutically acceptable salts thereof and mixtures thereof.

Exemplary anti-emetic active agents may include, without limitations, chlorpromazine, dimenhydrinate, dolasetron, dronabinol, granisetron, meclizine, metocloproamide, ondansetron, perphenazine, prochlorperazine, promethazine, scopolamine, thiethylperazine, trimethobenzamide, pharmaceutically acceptable salts thereof and mixtures thereof

### Excipients

In some embodiments, the suppositories described herein may further comprise a pharmaceutically acceptable excipient or carriers. The term “pharmaceutically acceptable excipient or carrier” refers to any inert ingredient in a composition that may act, for example, to stabilize the active ingredient. A pharmaceutically acceptable excipient can include, but is not limited to, carbohydrates (such as glucose, sucrose, or dextrans), antioxidants (such as d-α-tocopherol, ascorbic acid, or glutathione), chelating agents, low-molecular weight proteins, high-molecular weight polymers, gel-forming agents or other stabilizers and additives. Other examples of a pharmaceutically acceptable carrier include wetting agents (e.g., lecithin), emulsifying agents, surfactant and/or dispersing agents (e.g., polysorbate 80), alkalizing agents, coloring agents, synthetic dies, fillers, diluents, mineral oxides, or preservatives, which are particularly useful for preventing the growth or action of microorganisms. Various preservatives are well known and include, for example, phenol and ascorbic acid. Examples of carriers, stabilizers or adjuvants can be found in Remington's Pharmaceutical Sciences, Mack Publishing Company, Philadelphia, Pa., 17th ed. (1985).

Suitable antioxidants may include, without limitations, sterically hindered phenols, aryl amines, thioureas, thiocarbamates, phosphites, thioether esters, and combinations of the foregoing. Other suitable examples of antioxidants include, but are not limited to, alkylated monophenols, including but not limited to, 2,6-di-tert-butyl-4-methylphenol, 2-tert-butyl-4,6-di-methylphenol, 2,6-di-tert-butyl-4-ethylphenol, 2,6-di-tert-butyl-4-n-butylphenol, 2,6-di-tert-butyl-4-isobutylphenol, 2,6-dicyclopentyl-4-methylphenol, 2-(α-methylcyclohexyl)-4,6-dimethylphenol, 2,6-dioctadecyl-4-methylphenol, 2,4,6-tricyclohexylphenol, 2,6-di-tert-butyl-4-methoxymethylphenol, nonylphenols which are linear or branched in the side chains, for example, 2,6-di-nonyl-4-methylphenol, 2,4-dimethyl-6-(1′-methylundec-1′-yl)phenol, 2,4-dimethyl-6-(1′-methylheptadec-1′-yl)phenol, 2,4-dimethyl-6-(1′-methyltridec-1-yl)phenol and mixtures thereof, alkylthiomethylphenols, including but not limited to, 2,4-dioctylthiomethyl-6-tert-hutylphenol, 2,4-dioctylthiomethyl-6-methylphenol, 2,4-dioetylthiomethyl-6-ethylphenol, 2,6-di-dodecylthiornethyl-4-nonylphenol, hydroquinones and alkylated hydroquinones, including but not limited to, 2,6-di-tert-hutyl-4-methoxyphenol, 2,5-di-tert-butylhydroquinone, 2,5-di-tort-amylhydroquinone, 2,6-diphenyl-4-octadecyloxyphenol, 2,6-di-tert-butylhydroquinone, 2,5-di-tert-butyl-4-hydroxyanisole, 3,5-di-tert-butyl-4-hydroxyanisole, 3,5-di-tert-butyl-4-hydroxyphenyl stearate, bis(3,5-di-tert-butyl-4-hydroxyphenyl) adipate, tocopherols, including but not limited to, α-tocopherol, β-tocopherol, γ-tocopherol, δ-tocopherol and mixtures thereof (vitamin E), hydroxylated thiodiphenyl ethers, including but not limited to, 2,2′-thiobis(6-tort-butyl-4-methylphenol), 2,2′-thiobis(4-oetylphenol), 4,4′-thiobis(6-tert-butyl-3-methylphenol), 4,4′-thiobis(6-tert-butyl-2-methylphenol), 4,4′-thiobis(3,6-di-sec-amylphenol), 4,4′-bis(2,6-dimethyl-4-hydroxyphenyl)-disulfide, alkylidenebisphenols, including but not limited to, 2, 2′-methylenebis(6-tert-butyl-4-methylphenol), 2,2′-methylenebis(6-tert-butyl-4-ethylphenol), 2,2′-methylenebis[4-methyl-6-(α-methylcyclohexyl)-phenol], 2,2′-methylenebis(4-methyl-6-cyclohexylphenol), 2,2′-methylenebis(6-nonyl-4-methylphenol), 2,2′-methylenebis(4,6-di-tert-butylphenol), 2,2′-ethylidenebis(4,6-di-tert-butylphenol), 2,2′-ethylidenebis(6-tert-butyl-4-isobutylphenol), 2,2′-methylenebis[6-(α-methylbenzyl)-4-nonylphenol], 2,2′-methylenebis[6-(α,α-dimethylbenzyl)-4-nonylphenol], 4,4′-methylenebis(2,6-di-tert-butylphenol), 4,4′-methylenebis(6-tert-butyl-2-methylphenol), 1,1-bis(5-tert-butyl-4-hydroxy-2-methylphenyl)butane, 2,6-bis(3-test-butyl-5-methyl-2-hydroxybenzyl)-4-methylphenol, 1,1,3-tris(5-tert-butyl-4-hydroxy-2-methylphenyl)butane, 1,1-bis(5-tert-butyl-4-hydroxy-2-methyl-phenyl)-3-n-dodecylmercaptobutane, ethylene glycol bis[3,3-bis(3′-tert-butyl-4′-hydroxyphenyl)butyrate], bis(3-tert-butyl-4-hydroxy-5-methyl-phenyl)dicyclopentadiene, bis[2-(3′-tert-butyl-2′-hydroxy-5′-methylbenzyl)-6-tert-butyl-4-methylphenyl]terephthalate, 1,1-bis-(3,5-dimethyl-2-hydroxyphenyl)butane, 2,2-bis(3,5-di-tert-butyl-4-hydroxyphenyl)propane, 2,2-bis(5-tert-butyl-4-hydroxy-2-methylphenyl)-4-n-dodecylmercaptobutane, 1,5,5-tetra-(5-tert-butyl-4-hydroxy-2-methylphenyl)pentane, O-, N- and S-benzyl compounds, including but not limited to, 3,5,3′,5′-tetra-tert-butyl.-4,4′-dihydroxydibenzyl ether, octadecyl-4-hydroxy-3,5-dimethylbenzylmercaptoacetate, tridecyl-4-hydroxy-3,5-di-tert-butylbenzylmercaptoacetate, tris(3,5-di-tert-butyl-4-hydroxybenzyl)amine, bis(4-tert-butyl-3-hydroxy-2,6-dimethylbenzyl)dithioterephthalate, bis(3,5-di-tert-butyl-4-hydroxybenzyl)sulfide, isooctyl-3,5-di-tert-butyl-4-hydroxybenzylmercaptoacetate, hydroxybenzylated malonates, including but not limited to, dioctadecyl-2,2-bis(3,5-di-tert-butyl-2-hydroxybenzyl)malonate, di-octadecyl-2-(3-tert-butyl-4-hydroxy-5-methylbenzyl)malonate, didodecylmercaptoethyl-2,2-bis(3,5-di-tert-butyl-4-hydroxybenzyl)malonate, bis[4-(1,1,3,3-tetramethylbutyl)phenyl]-2,2-bis(3,5-di-tert-butyl-4-hydroxybenzyl)malonate, aromatic hydroxybenzyl compounds, including but not limited to, 1,3,5-tris(3,5-di-tert-butyl-4-hydroxybenzyl)-2,4,6-trimethylbenzene, 1,4-bis(3,5-di-tert-butyl-4-hydroxybenzyl)-2,3,5,6-tetramethylbenzene, 2,4,6-tris(3,5-di-tert-butyl-4-hydroxybenzyl)phenol, triazine compounds, including but not limited to, 2,4-bis(octylmercapto)-6-(3,5-di-tert-butyl-4-hydroxyanilino)-1,3,5-triazine, 2-octylmercapto-4,6-bis(3,5-di-tert-butyl-4-hydroxyanilino)-1,3,5-triazine, 2-octylmercapto-4,6-bis(3,5-di-tert-butyl-4-hydroxyphenoxy)-1,3,5-triazine, 2,4,6-tris-(3,5-di-tert-butyl-4-hydroxyphenoxy)-1,2,3-triazine, 1,3,5-tris(3,5-di-tert-butyl-4-hydroxybenzyl)isocyanurate, 1,3,5-tris(4-tert-butyl-3-hydroxy-2,6-dimethylbenzyl)isocyanurate, 2,4,6-tris-(3,5-di-tert-butyl-4-hydroxyphenylethyl)-1,3,5-triazine, 1,3,5-tris(3,5-di-tert-butyl-4-hydroxy-phenylpropionyl)-hexahydro-1,3,5-triazine, 1,3,5-tris(3,5-dicyclohexyl-4-hydroxybenzyl)isocyanurate, benzylphosphonates, including but not limited to, dimethyl-2,5-di-tert-butyl-4-hydroxybenzylphosphonate, diethyl-3,5-di-tert-butyl-4-hydroxybenzylphosphonate, dioctadecyl3,5-di-tent-butyl-4-hydroxybenzylphosphonate, dioctadecyl-5-tert-butyl-4-hydroxy-3-methylbenzylphosphonate, the calcium salt of the monoethyl ester of 3,5-di-tert-butyl-4-hydroxybenzylphosphonic acid, acylaminophenols, including but not limited to, 4-hydroxylauranilide, 4-hydroxystearanilide, octyl N-(3,5-di-tert-butyl-4-hydroxyphenyl)carbamate, esters of β-(3,5-di-tert-butyl-4-hydroxyphenyl)propionic acid with mono- or polyhydric alcohols, e.g. with methanol, ethanol n-octanol, i-octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl)isocyanurate, N,N′-bis(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2.2]octane, esters of β-(5-tert-butyl-4-hydroxy-3-methylphenyl)propionic acid with mono- or polyhydric alcohols, e,g. with methanol, ethanol, n-octanol, i-octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl)isocyanurate, N,N′-bis-(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2.2] octane; 3,9-bis[2-{3-(3-tert-butyl-4-hydroxy-5-methylphenyl)propionyloxy}-1,1-dimethylethyl]-2,4,8,10-tetraoxaspiro[5.5]-undecane, esters of 6-(3,5-dicyclohexyl-4-hydroxyphenyl)propionic acid with mono- or polyhydric alcohols, e.g. with methanol, ethanol, octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl)isocyanurate, N,N′-bis(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2,2]octane, esters of 3,5-di-tert-butyl-4-hydroxyphenyl acetic acid with mono- or polyhydric alcohols, e.g. with methanol, ethanol, octanol, octadecanol, 1,6-hexanediol, 1,9-nonanediol, ethylene glycol, 1,2-propanediol, neopentyl glycal, thiodiethyl.ene glycol, diethylene glycol, triethylene glycol, pentaerythritol, tris(hydroxyethyl)isocyanurate, N,N′-bis(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2.2]octane, amides of 6-(3,5-di-tert-butyl-4-hydroxyphenyl)propionic acid e.g. N,N′-bis(3,5-di-tert-butylA-hydroxyphenylpropionyl)hexamethylenediamide, N,N′-bis(3,5-di-tert-butyl-4-hydroxyphenylpropionyl)trimethylenediamide, N,N′-bis(3,5-di-tert-butyl-4-hydroxyphenylpropionyl)hydrazide, N,N′-bis[2-(3-[3,5-di-tert-butyl-4-hydroxyphenyl]propionyloxy)ethyl]oxamide (Naugard®XL-1, supplied by Uniroyal), ascorbic acid (vitamin C), aminic antioxidants, including but not limited to, N,N′-di-isopropyl-p-phenylenediamine, N,N′-di-sec-butyl-p-phenylenediamine, N,N′-bis(1,4-dimethylpentyl)-p-phenylenediamine, N,N′-bis(1-ethyl-3-methylpentyl)-p-phenylenediamine, N,N′-bis(1-methylheptyl)-p-phenylenediamine, N,N′-dicyclohexyl-p-phenylenediamine, N,N′-diphenyl-p-phenylenediamine, N,N′-bis(2-naphthyl)-p-phenylenediamine, N-isopropyl-N′-phenyl-p-phenylenediamine, N-(1,3-dimethylbutyl)-N′-phenyl-p-phenylenediamine, N-(1-methylheptyI)-N′-phenyl-p-phenylenediamine, N-cyclohexyl-N′-phenyl-p-phenyienediamine, 4-(p-toluenesulfamoyl)diphenylamine, N,N′-dimethyl-N,N′-di-sec-butyl-p-phenylenediamine, diphenylamine, N-allyldiphenylamine, 4-isopropoxydiphenylamine, N-phenyl-1-naphthylamine, N-(4-tert-octylphenyl)-1-naphthylamine, N-phenyl-2-naphthylamine, octylated diphenylamine, including but not limited to, p,p′-di-tert-octyldiphenylamine, 4-n-butylaminophenol, 4-butyrylaminophenol, 4-nonanoylaminophenol, 4-dodecanoylaminophenol, 4-octadecanoylaminophenol, bis(4-methoxyphenyl)amine 2,6-di-tert-butyl-4-dimethylaminomethylphenol, 2,4′-diaminodiphenylmethane, 4,4′-diaminodiphenylmethane, N,N,N′,N′-tetramethyl-4,4′-diaminodiphenylmethane, 1,2-bis[(2-methylphenyl)amino]ethane, 1,2-bis(phenylamino)propane, (o-tolyl)biguanide, bis[4-(1′,3′-dimethylbutyl)phenyl]amine, tert-octylated N-phenyl-1-naphthylamine, a mixture of mono- and dialkylated tert-butyl/tert-octyldiphenylamines, a mixture of mono- and dialkylated nonyldiphenylamines, a mixture of mono- and dialkylated dodecyldiphenylamines, a mixture of mono- and dialkylated isopropyl/isohexyldiphenylamines, a mixture of mono- and dialkylated teak-butyldiphenylamines, 2,3-dihydro-3,3-dimethyl-4H-1,4-benzothiazine, phenothiazine, a mixture of mono- and dialkylated tert-butyl/tert-octylphenothiazines, a mixture of mono- and dialkylated tert-octyl-phenothiazines, N-allylphenothiazine, N,N,N′,N′-tetraphenyl-1,4-diaminobut-2-ene, and combinations of the foregoing.

Suitable alkalizing agents may include, without limitations, magnesium oxide, ammonium hydroxide, sodium hydroxide, sodium carbonate, sodium citrate, trisodium phosphate and/or disodium phosphate.

Suitable coloring agents may include, but are not limited to, colors such as e.g., white, black, yellow, blue, green, pink, red, orange, violet, indigo, and brown. In specific embodiments, the color of the dosage form can indicate the contents (e.g., one or more active agents) contained therein.

Suitable lubricants/release agents can include, but are not limited to, fatty acids and their salts, fatty alcohols, fatty esters, fatty amines, fatty amine acetates and fatty amides. Other suitable lubricants may include, but not be limited to, glyceryl behenate (Compritol™ 888), metallic stearates (e.g., magnesium, calcium and sodium stearates), stearic acid, hydrogenated vegetable oils (e.g., Sterotex™), talc, waxes such as beeswax and carnauba wax, silica, fumed silica, colloidal silica, calcium stearate, long chain fatty alcohols, boric acid, sodium benzoate and sodium acetate, sodium chloride, DL-Leucine, polyethylene glycols (e.g., Carbowax™ 4000 and Carbowax™ 6000), sodium oleate, sodium benzoate, sodium acetate, sodium lauryl sulfate, sodium stearyl fumarate (Pruv™), magnesium lauryl sulfate, stearic acid, stearyl alcohol, mineral oil, paraffin, micro crystalline cellulose, glycerin, propylene glycol and combinations thereof.

Suitable extenders/antiblocking agents/detackifying agents can include, but are not limited to, starches, modified starches, crosslinked polyvinylpyrrolidone, crosslinked cellulose, microcrystalline cellulose, silica, metallic oxides, calcium carbonate, talc and mica.

Suitable diluents include, but are not limited to, lactose USP, lactose USP (anhydrous), lactose USP (spray dried), starch USP, directly compressible starch, mannitol USP, sorbitol, dextrose monohydrate, microcrystalline cellulose NF, dibasic calcium phosphate dihydrate NF, sucrose-based diluents, confectioner's sugar, monobasic calcium sulfate monohydrate, calcium sulfate dihydrate NF, calcium lactate trihydrate granular NF, dextrates NF (e.g., Emdex™), dextrose (e.g., Cerelose™), inositol, hydrolyzed cereal solids such as the Maltrons™ and Mor-Rex™, amylose, powdered cellulose (e.g., Elcema™) calcium carbonate, glycine, bentonite, polyvinylpyrrolidone, and the like.

Exemplary oils and fats may include, but not be limited to, almond oil, argan oil, avocado oil, canola oil, cashew oil, castor oil, cocoa butter, coconut oil, colza oil, corn oil, cottonseed oil, grape seed oil, hazelnut oil, hemp oil, hydroxylated lecithin, lecithin, linseed oil, macadamia oil, mango butter, manila oil, mongongo nut oil, olive oil, palm kernel oil, palm oil, peanut oil, pecan oil, perilla oil, pine nut oil, pistachio oil, poppy seed oil, pumpkin seed oil, rice bran oil, safflower oil, sesame oil, shea butter, soybean oil, sunflower oil, walnut oil, and watermelon seed oil. Other oil and fats that may be in the fill of the PVA shell may include, but not be limited to, fish oil (omega-3), crill oil, animal or vegetable fats, e.g., in their hydrogenated form, mono-, di-, and tri-glycerides with C12-, C14-, C16-, C18-, C20- and C22-fatty acids.

Exemplary pH modifiers may include, but not be limited to, hydrochloric acid, potassium hydroxide, sodium hydroxide, ammonium hydroxide, sulfuric acid, phosphoric acid, and nitric acid.

Other exemplary excipients may include, but not be limited to, vegetable proteins such as sunflower protein, soybean proteins, cotton seed proteins, peanut proteins, grape seed proteins, whey proteins, whey protein isolates, blood proteins, egg proteins, acrylated proteins, water-soluble polysaccharides such as alginates, carrageenans, guar gum, agar-agar, xanthan gum, gellan gum, gum arabic and related gums (gum ghatti, gum karaya, gum tragancanth), pectin, water-soluble derivatives of cellulose: alkylcelluloses hydroxyalkylcelluloses and hydroxyalkylalkylcelluloses, such as methylcelulose, hydroxymethylcellulose, hydroxyethylcellulose, hydroxypropylcellulose, hydroxyethylmethylcellulose, hydroxypropylmethylcellulose, hydroxybutylmethylcellulose, cellulose esters and hydroxyalkylcellulose esters such as cellulose acetate phthalate (CAP), hydroxypropylmethylcellulose (HPMC); carboxyalkylcelluloses, carboxyalkylalkylcelluloses, carboxyalkylcellulose esters such as carboxymethylcellulose and their alkali metal salts; water-soluble synthetic polymers such as polyacrylic acids, polyacrylamides, and polyacrylic acid esters, polymethacrylic acids, polymethacrylamides, and polymethacrylic acid esters, polyvinylacetates, polyvinylalcohols, polyvinylacetatephthalates (PVAP), polyvinylpyrrolidone (PVP), PVY/vinyl acetate copolymer, and polycrotonic acids; also suitable are phthalated gelatin, gelatin succinate, crosslinked gelatin, shellac, water-soluble chemical derivatives of starch, cationically modified acrylates and methacrylates possessing, for example, a tertiary or quaternary amino group, such as the diethylaminoethyl group, which may be quaternized if desired; and other similar polymers; inorganic fillers, such as the oxides of magnesium aluminum, silicon, titanium, etc.

Other pharmaceutically acceptable excipients may include, without limitations, a hydrophobic material, including, but is not limited to, digestible, long chain (C₈-C₅₀, especially C₁₂-C₄₀), substituted or unsubstituted hydrocarbons, such as natural or synthetic waxes (such as beeswax, glycowax, castor wax and carnauba wax), fatty alcohols (such as lauryl, myristyl, stearyl, cetyl or preferably cetostearyl alcohol), fatty acids, including, but not limited to, mono-diglyceride of medium chain fatty acids (such as caprylic, capric, caproic, lauric, oleic, linoleic), medium chain triglycerides, fatty acid esters, fatty acid glycerides (mono-, di-, and tri-glycerides), hydrogenated fats, hydrocarbons, normal waxes, stearic acid, stearyl alcohol and hydrophobic and hydrophilic materials having hydrocarbon backbones.

Additional pharmaceutically acceptable excipients may further include polyvinyl alcohols, polyvinyl pyrrolidone, polyalkylene oxides, polyacrylic acid, cellulose, cellulose ethers, cellulose esters, cellulose amides, polyvinyl acetates, polycarboxylic acids and salts, acetic acid, caprylic acid, oleic acid, polyaminoacids or peptides, polyamides, polyacrylamide, copolymers of maleic/acrylic acids, polysaccharides including starch and gelatin, natural gums such as xanthan, and carrageenans. For example, polymers can be selected from polyacrylates and water-soluble acrylate copolymers, methylcellulose, carboxymethylcellulose sodium, dextrin, ethylcellulose, hydroxyethyl cellulose, hydroxypropyl methylcellulose, maltodextrin, polymethacrylates, and combinations thereof, or selected from polyvinyl alcohols, polyvinyl alcohol copolymers and hydroxypropyl methyl cellulose (HPMC), methacrylic acid/methyl methacrylate, methacrylic acid/ethyl acrylate copolymers, methacrylic acid/methyl acrylate/methyl methacrylate copolymers, shellac, hydroxypropyl methylcellulose phthalate, hydroxyl propyl methyl cellulose acetate succinate, hydroxypropyl methyl cellulose trimellitate, cellulose acetate phthalates, polyvinyl acetate phthalates, PEG-35 castor oil, caprylocaproyl polyoxyl-8 glycerides, glyceryl distearate, and combinations thereof.

Suitable high HLB surfactants may include, without limitations, polysorbate 80-polyoxyethylene (20) sorbitan monooleate, polyoxyl 40 hydrogenated castor oil, polyoxyl 35 castor oil, caprylocaproyl macrogol glycerides, and combinations thereof.

Exemplary fillers may be selected, without limitations, from the group consisting of lactose, microcrystalline cellulose, and combinations thereof.

In some embodiments, the excipients may independently or cumulatively be present in the suppository at a concentration of about 90 w/w % or less, about 80 w/w % or less, about 70 w/w % or less, about 60 w/w % or less, about 50 w/w % or less, about 40 w/w % or less, about 30 w/w % or less, about 20 w/w % or less, about 15 w/w % or less, about 10 w/w % or less, about 5 w/w % or less, about 4 w/w % or less, about 3 w/w % or less, about 2 w/w % or less, about 1 w/w % or less, about 0.5 w/w % or less, about 0.1 w/w % or less, based on the total weight of the suppository. In some embodiments, the suppository may comprise excipients in an amount ranging, e.g., from about 2 w/w % to about 50 w/w %, from about 6 w/w % to about 40 w/w %, from about 10 w/w % to about 30 w/w %, from about 10 w/w % to about 40 w/w %, from about 15 w/w % to about 35 w/w %, from about 20 w/w % to about 30 w/w %, from about 20 w/w % to about 25 w/w %, or from about 15 w/w % to about 25 w/w %, individually or collectively, based on the total weight of the suppository.

### Method of Preparation

In some embodiments, the instant disclosure may be directed to a method of preparing any of the suppositories described herein. The method may comprise forming a suppository base into a suppository. With respect to FIGS. 1-7, the suppository may be formed to have a length 110, measured along horizontal axis X, and a width 120, measured along a vertical axis Y, such that the length 110 is greater than the width 120. The suppository may be formed to have a proximal end 160 and a distal end 170 along the horizontal axis X. The proximal end 160 may be formed to be adapted for insertion into a body cavity. The distal end 170 may be formed with at least one indentation for easy grip of the suppository such that the suppository is less likely to slip away upon manual insertion of the suppository into a body cavity (whether the manual insertion is done with the fingers or with an applicator). The presence of the “grip area” on the distal end of the suppository points to the direction in which the suppository should be used (i.e., for the proximal end to enter the body cavity first). Furthermore, the shape of the “grip area” in the distal end of the suppository makes it less likely to be expelled from the body cavity.

Similarly, the method may comprise forming a suppository base into a suppository having the dimensions (lengths and/or diameters) and proximal and distal shapes as depicted with respect to FIGS. 8-35 described in further detail hereinabove.

The method of preparing any of suppositories described herein may further comprise dispersing any of the active agents contemplated herein in the suppository base. In some embodiments, the active agent may be dispersed throughout the suppository base in its entirety. In other embodiments, the active agent may be dispersed only in a portion of the suppository base. For example, in an embodiment, the active agent may not be dispersed at least in a portion of the distal end of the suppository base. In embodiments where a core and a shell structure suppository is prepared, the method of preparation may comprise dispersing the active agent in the core only, the shell only, or in both—the core and the shell.

In certain embodiments, the method of preparing a suppository may further comprise applying a shell onto the suppository base to form a core and a shell structure. In one embodiment, a core and shell structure suppository may be prepared by dipping a core in a shell composition. For instance, the core may be solid at ambient temperature and may be dipped into a liquid shell composition.

In one embodiment, a core and shell structure suppository may be prepared by encapsulating a liquid comprising the suppository base in the shell. For instance, the core may be liquid and may be encapsulated by stamping (such as rotary die stamping). The encapsulation may be performed at ambient temperature or above ambient temperature. For example, the encapsulation may be performed at a temperature that is above ambient temperature when the core is liquid for easy processing and handling. In certain embodiments, a core that is liquid at an encapsulation temperature that is above ambient temperature may solidify at ambient temperature and may melt again upon insertion into a body cavity (i.e., upon exposure to a human core temperature).

In some embodiments, the suppository base may encapsulate a suppository base (comprising an active agent) using rotary die stamping/encapsulation. Encapsulation of the suppository base by rotary die encapsulation may use conventional rotary die equipment with molds that are designed to form an indented suppository capsule shape such as the ones described herein. Similarly, in a core and shell suppository structure, encapsulation of the core in a shell by rotary die encapsulation may use conventional rotary die equipment with molds that are designed to form an indented suppository capsule shape such as the ones described herein.

According to an embodiment, any of the suppository capsules disclosed herein may be prepared by a process comprising the steps of: (a) preparing the suppository base; (b) combining the suppository base of step (a) with an active agent to form a core; and optionally (c) applying a shell composition onto a core comprising the combined suppository base and active agent.

Preparing the suppository base according to step (a) may comprise mixing a plurality of pharmaceutically acceptable excipients or carriers.

In certain embodiments, step (b) may comprise mixing the active agent with the suppository base. In other embodiments, step (b) may comprise encapsulating the active agent in a suppository base using rotary die encapsulation process to form a filled suppository base in an indented suppository shape.

In certain embodiments, step (c) may comprise dipping the core from step (b) into a pre-mixed shell composition. In other embodiments, step (c) may comprise encapsulating the core from step (b) in a shell composition using rotary die encapsulation process to form an indented suppository shape having a core and shell structure.

### Method of Treatment

In some embodiments, the instant disclosure may be directed to a method of treating a disease or a condition by administering any of the suppositories described herein into a body cavity of a patient in need thereof.

Administering the suppository may comprise gripping/holding the suppository in the at least one indentation on the distal end of the suppository. Gripping/holding the suppository may be done with at least one finger. In some embodiments, gripping the suppository may be done with two fingers, with three fingers, with four fingers, or with five fingers. In certain embodiments, gripping the suppository may be done with a tool, such as an applicator, forceps, and the like. In other embodiments, gripping the suppository may be done without any tools such as an applicator, forceps, and so on.

In certain embodiments, administering the suppository may further comprise inserting (e.g., manually) the suppository into a body cavity via the proximal end direction (i.e. such that the proximal end is inserted into the body cavity first). The body cavity may be, without limitations, the rectum, the vagina, or the urethra and the suppository may be adapted for vaginal insertion, for rectal insertion, or for urethral insertion accordingly.

Exemplary disease or conditions that may be treated with any of the suppositories described herein may include, without limitations, pain, migraine, inflammation, fungal infection, bacterial infection, viral infection, vaginal pain, menopause, vulvar and vaginal atrophy, cancer, nausea, vomiting, or a combination thereof.

The suppositories described herein may provide a local therapeutic effect, a systemic therapeutic effect, or a both—a local and a systemic therapeutic effect.

In the foregoing description, numerous specific details are set forth, such as specific materials, dimensions, processes parameters, etc., to provide a thorough understanding of the present invention. The particular features, structures, materials, or characteristics may be combined in any suitable manner in one or more embodiments. The words “example” or “exemplary” are used herein to mean serving as an example, instance, or illustration. Any aspect or design described herein as “example” or “exemplary” is not necessarily to be construed as preferred or advantageous over other aspects or designs. Rather, use of the words “example” or “exemplary” is simply intended to present concepts in a concrete fashion. As used in this application, the term “or” is intended to mean an inclusive “or” rather than an exclusive “or”. That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. Reference throughout this specification to “an embodiment”, “certain embodiments”, or “one embodiment” means that a particular feature, structure, or characteristic described in connection with the embodiment is included in at least one embodiment. Thus, the appearances of the phrase “an embodiment”, “certain embodiments”, or “one embodiment” in various places throughout this specification are not necessarily all referring to the same embodiment.

The present invention has been described with reference to specific exemplary embodiments thereof. The specification and drawings are, accordingly, to be regarded in an illustrative rather than a restrictive sense. Various modifications of the invention in addition to those shown and described herein will become apparent to those skilled in the art and are intended to fall within the scope of the appended claims.

1. A suppository comprising a suppository base, the suppository having a length, measured along a horizontal axis, greater than a width, measured along a vertical axis, the suppository having a proximal end along the horizontal axis adapted for insertion into a body cavity and a distal end along the horizontal axis having at least one indentation.

2. The suppository of claim 1, further comprising an active agent.

3. The suppository of claim 2, wherein the active agent is dispersed in the suppository base.

4. The suppository of claim 2, wherein the active agent is dispersed throughout the suppository base in its entirety.

5. The suppository of claim 2, wherein the active agent is not dispersed throughout the suppository base in at least a portion of the distal end.

6. The suppository of claim 2, wherein the distal end has two indentations.

7. The suppository of claim 6, wherein each indentation is on opposing sides of the distal end.

8. (canceled)

9. The suppository of claim 1, having a shape selected from an indented bullet, an indented oval, an indented ovoid, an indented teardrop, or an indented torpedo.

10. The suppository of claim 1, wherein the at least one indentation is flat, concave or perforated.

11. (canceled)

12. (canceled)

13. The suppository of claim 1, having a core and shell structure.

14. The suppository of claim 13, wherein an active agent is contained in the core, the shell, or in the core and the shell.

15. The suppository of claim 13, wherein the core comprises the suppository base.

16. The suppository of claim 1, wherein the shell comprises at least one of gelatin, starch, modified starch, carrageenan, alginate, a biodegradable polymer, or a combination thereof.

17. (canceled)

18. The suppository of claim 1, wherein the suppository base comprises at least one of cocoa butter, lauric oil, beef tallow, hard fat, theobroma oil, glycerides of fatty acids, glycerol-gelatin bases, or a combination thereof.

19. (canceled)

20. (canceled)

21. (canceled)

22. The suppository of claim 1, adapted for vaginal insertion, rectal insertion, or urethral insertion.

23. The suppository of claim 1, providing a local therapeutic effect.

24. The suppository of claim 1, any one of the preceding claims, providing a systemic therapeutic effect.

25. The suppository of claim 2, wherein the active agent is selected from an analgesic, anti-migraine, anti-inflammatory, anti-fungal, antibiotic, anti-viral, hormone, chemotherapeutic, anti-nausea, or anti-emetic.

26.-40. (canceled)

41. The suppository of claim 16, wherein the shell composition further comprises a plasticizer.

42.-44. (canceled)

45. The suppository of claim 16, wherein the shell composition comprises about 40 wt % to about 80 wt %, about 45 wt % to about 75 wt %, or about 50 wt % to about 70 wt %, of gelatin, based on total weight of the shell composition.

46. The suppository of claim 41, wherein the shell composition comprises about 15 wt % to about 40 wt %, about 20 wt % to about 35 wt %, or about 25 wt % to about 30 wt % of plasticizer, based on total weight of the shell composition.

47. A method of treating a disease or condition comprising inserting a suppository of claim 1 into a body cavity of a patient in need thereof.

48.-50. (canceled)

51. A method of preparing a suppository comprising forming a suppository base into a suppository, the suppository having a length, along a horizontal axis, greater than a width, along a vertical axis, the suppository having a proximal end along the horizontal axis adapted for insertion into a body cavity and a distal end along the horizontal axis having at least one indentation.

52. The method of claim 51, further comprising dispersing an active agent in the suppository base.

53. The method of claim 51, comprising applying a shell onto the suppository base to form a core and shell structure.

54.-61. (canceled)
